(19) United States
(12) Patent Application Publication
(10) Pub. No.: US 2026/0313317 A1
(43) Pub. Date: Oct. 8, 2026

(54) WIRELESS TAGS FOR ADVANCED HEALTHCARE ARTICLE TRACKING SYSTEM

(71) Applicant: Peritas AI Inc., Fort Lauderdale, FL (US)

(72) Inventors: Martin Roche, Fort Lauderdale, FL (US); Joseph DeCerce, Fort Lauderdale, FL (US)

(21) Appl. No.: 19/566,805

(22) Filed: Mar. 13, 2026

Related U.S. Application Data
(63) Continuation-in-part of application No. 19/431,214, filed on Dec. 23, 2025.
(60) Provisional application No. 63/782,696, filed on Apr. 3, 2025.

(51) Int. Cl. H04W 4/02; G06K 19/077; G16H 40/40; H04W 4/80

(52) U.S. Cl. CPC H04W 4/027; G06K 19/0776; G16H 40/40; H04W 4/80

(57) ABSTRACT

Various sensing technologies may be used together for an advanced healthcare article tracking system to track items within a hospital. Asset tracking may therefore be implemented to provide both humans and robots the ability to track assets as they are stored and moved about within the hospital, with the system also optimizing the scheduling and utilization of operating rooms based on the tracking of the hospital's assets. In one particular instance, Bluetooth tags or other wireless tags may be implemented to allow the tracking of hospital assets throughout the hospital.

### FIELD

The present application relates generally to wireless tags such as Bluetooth tags for advanced healthcare article tracking systems that use near-field technologies.

### BACKGROUND

Tracking assets within a healthcare facility can be complex, with various assets often being misplaced, not ready for use, in an unknow location, or in short supply.

### SUMMARY

As understood herein, prior to surgery, for example, the necessary surgical instruments or tools required by the attending surgeon for a particular procedure must be sterilized, assembled into a tray, the tray sterilized, and then transported to the operating room (OR) in time for the scheduled surgery. This also requires significant coordination with OR cleaning and the provision of additional hospital assets such as prosthetics, patient surgical beds, etc. Post-surgery, assets must be returned to the appropriate places in the hospital and tools re-sterilized for subsequent use. Present principles understand that various tracking and artificial intelligence (AI) technologies may be highly advantageous for coordinating these things.

Accordingly, in one aspect a system includes a first Bluetooth (BT) tag storing a first identifier associated with a surgical case cart, where the first BT tag is configured to communicate with a plurality of BT readers spaced apart from each other in a healthcare facility. The system also includes a second BT tag storing a second identifier associated with a surgical bed, where the second BT tag is configured to communicate with the plurality of BT readers. The system further includes a third BT tag storing a third identifier associated with a medical implant, where the third BT tag is configured to communicate with the plurality of BT readers.

In certain example embodiments, each of the first, second, and third BT tags may include a respective motion sensor, and each of the first, second, and third BT tags may be configured to broadcast the respective first, second, or third identifier responsive to one or more signals from the respective motion sensor indicating motion of the respective BT tag. Also in certain instances, each respective motion sensor may include a respective accelerometer.

Also in non-limiting examples, each of the first, second, and third BT tags may be configured to broadcast the respective first, second, or third identifier at respective predefined intervals.

Still further, in some implementations the system may include a processor system and storage accessible to the processor system. The storage may include instructions executable by the processor system to identify a respective location of each of the first, second, and third BT tags based on receipt of one or more signals from each respective BT tag. The instructions may also be executable to present a graphical user interface (GUI) on a display, where the GUI may indicate the respective locations. In addition, in some specific instances the instructions may be executable to identify the respective locations using one or more of one or more channel sounding algorithms, one or more phased-based ranging algorithms, one or more round trip timing algorithms, and/or one or more received signal strength indicator (RSSI) algorithms. Also in some examples, the system may include the display, and/or the processor system and storage may be embodied on a server associated with the healthcare facility.

In addition, if desired the system may again include a processor system and storage accessible to the processor system. The storage may include instructions executable by the processor system to identify a respective location of one or more of the first, second, and third BT tags based on receipt of one or more signals from each respective BT tag and, based on a respective location of one or more of the first, second, and third BT tags, determine a time at which an operation on a patient is to start. Based on the determination of the time at which the operation is to start, the instructions may also be executable to transmit a respective notification to one or more client devices, where each respective notification may indicate the time at which the operation on the patient is to start. In some specific instances, the instructions may even be executable to, based on respective locations of the first BT tag and the third BT tag in particular, determine the time at which the operation on the patient is to start. What's more, in some specific cases the time may be a first time and here the instructions may be executable to, based on the respective locations of the first BT tag and the third BT tag, determine a second time at which the surgical case cart and the medical implant will be located at an operating room. Based on the determination of the second time at which the surgical case cart and the medical implant will be located at the operating room, the instructions may be executable to determine the first time at which the operation on the patient is to start. The first time may be the same as the second time, or the first time may be no more than a threshold amount after the second time. Also in certain situations, the second time may be an earliest time at which the surgical case cart and the medical implant will be located at the operating room as determined based on current movement of both the first BT tag and the third BT tag. What's more, each respective notification may indicate an identifier for the operating room.

In addition, in some specific embodiments the third BT tag may be a sticker tag.

What's more, in some cases the system may include the plurality of BT readers, and may further include an additional BT reader and cellular communication transceiver disposed on a vehicle that transports the medical implant to the healthcare facility.

Still further, in some cases the first BT tag may be embodied on a case cart tag that includes both the first BT tag and another tag different from a BT tag, where the other tag may include a transmitter configured to transmit wireless signals in a frequency range of 30-900 kHz.

In another aspect, a system includes a first wireless communication tag storing a first identifier associated with a surgical case cart, where the first wireless communication tag is configured to communicate with a plurality of readers spaced apart from each other in a healthcare facility. The system also includes a second wireless communication tag storing a second identifier associated with a medical implant, where the second wireless communication tag is configured to communicate with the plurality of readers. The system further includes a processor system and storage accessible to the processor system. The storage includes instructions executable by the processor system to receive one or more first signals from the first wireless communication tag, receive one or more second signals from the second wireless communication tag, and, based on the one or more first signals and the one or more second signals, update an inventory database for the healthcare facility. The instructions are also executable to receive one or more third signals from the first wireless communication tag, receive one or more fourth signals from the second wireless communication tag, and based on the one or more third signals and the one or more fourth signals, manage the scheduling of one or more operations in the healthcare facility.

In still another aspect, a method includes receiving one or more first signals from a first wireless communication tag, receiving one or more second signals from a second wireless communication tag, and based on the one or more first signals and the one or more second signals, updating an inventory database for a healthcare facility. The method also includes receiving one or more third signals from the first wireless communication tag, receiving one or more fourth signals from the second wireless communication tag, and based on the one or more third signals and the one or more fourth signals, managing the scheduling of one or more operations in the healthcare facility.

The details of the present application, both as to its structure and operation, can be best understood in reference to the accompanying drawings, in which like reference numerals refer to like parts, and in which:

### BRIEF DESCRIPTION OF THE DRAWINGS

FIG. 1 is a block diagram of an example overall system in accordance with present principles;

FIG. 2 is a schematic diagram of a tracking system consistent with present principles;

FIG. 3 is a schematic diagram of example tracking components that may be implemented consistent with present principles;

FIG. 4 shows another diagram of example tracking components that may be implemented consistent with present principles;

FIGS. 5 and 6 show exploded views of various components of an example autoclave tag that may be attached to a surgical tool tray consistent with present principles;

FIGS. 7-9A show various perspective views of the autoclave tag consistent with present principles;

FIGS. 9B-9E show various example cutaway views of the autoclave tag consistent with present principles;

FIG. 10 shows a perspective view of the autoclave tag as affixed to a surgical tool tray consistent with present principles;

FIG. 11 shows a close-up view of the autoclave tag as affixed to the surgical tool tray consistent with present principles;

FIG. 12 shows a close-up view of the back of the autoclave tag as attached to the surgical tool tray consistent with present principles;

FIG. 13 shows a perspective partial view of the autoclave tag attached to the surgical tool tray and the tray placed within a case cart consistent with present principles;

FIG. 14 shows a perspective view of example autoclave tag readers consistent with present principles;

FIG. 15 shows a perspective view of two storage racks with tool trays the presence of which may be monitored by an autoclave tag reader consistent with present principles;

FIGS. 16 and 17 show perspective views of a case cart transporting plural surgical tool trays and exiting a room through a doorway while an autoclave tag reader registers the presence of the tool trays consistent with present principles;

FIG. 18 shows a side elevational view of a surgical tool tray with an autoclave tag attached and with the tray located on a storage rack shelf consistent with present principles;

FIG. 19 shows a diagram of an example arrangement in which a field axis of an autoclave tag is aligned with the axis of an antenna of an autoclave tag reader consistent with present principles;

FIG. 20 shows a perspective of various autoclave tag transmitting and receiving components as aligned to optimize communication consistent with present principles;

FIG. 21 illustrates example logic in flowchart format that may be executed by an IC of an autoclave tag consistent with present principles;

FIG. 22 illustrates example logic in flowchart format that may be executed by an IC of an autoclave tag reader consistent with present principles;

FIG. 23 shows an exploded view of an example Bluetooth (BT) or other longer-range wireless signal reader that may be implemented consistent with present principles;

FIG. 24 shows a perspective transparent view of the BT reader consistent with present principles;

FIGS. 25-28 shows various perspective views of the BT reader consistent with present principles;

FIG. 29 shows a perspective view of an example mounting bracket for the BT reader consistent with present principles;

FIG. 30 shows a perspective view of example BT tags that may be used consistent with present principles;

FIG. 31 shows a perspective view of the example BT tags as affixed to various healthcare-related articles of a healthcare facility consistent with present principles;

FIG. 32 illustrates example logic in flowchart format that may be executed by an IC in a BT tag consistent with present principles;

FIG. 33 illustrates example logic in flowchart format that may be executed by an IC in a BT tag reader consistent with present principles;

FIG. 34 shows an example software feature tree for software that may be implemented consistent with present principles;

FIG. 35 is an illustration of an example hospital layout with tracking components consistent with present principles;

FIG. 36 is an illustration of an example sterile processing department with tracking components consistent with present principles;

FIG. 37 shows an example graphical user interface (GUI) that may be presented on a display of a client device for hospital personnel to view the real-time locations of various articles consistent with present principles;

FIG. 38 shows an example GUI that may be presented on a display of a client device to alert hospital personnel that certain surgical tool trays contain unsterile tools and/or a wet load of tools consistent with present principles;

FIG. 39 shows two example case carts demonstrating that autoclave tags can be read through open and closed carts consistent with present principles;

FIG. 40 shows blue-wrapped surgical tool trays in an SPD and autoclave tags attached to the blue-wrapping consistent with present principles;

FIG. 41 shows a perspective view of orange clips for surgical tool trays as also including autoclave tag antennas consistent with present principles;

FIGS. 42-45 show various views of an autoclave tag reader that may be coupled to a smartphone or other client device consistent with present principles;

FIGS. 46A and 46B show example GUIs that may be presented on the display of the smartphone based on its autoclave tag reader reading certain autoclave tags consistent with present principles;

FIG. 47 shows a robot in an SPD locating a blue-wrapped tray using an on-board autoclave tag reader and computer vision consistent with present principles; and

FIGS. 48A-48C show various views demonstrating that different parts of an operating room may also be equipped with autoclave tag reader antennas to monitor items during and immediately after surgery consistent with present principles.

### DETAILED DESCRIPTION

The disclosure below generally relates to healthcare article tracking systems using near-field technologies to improve the tracking, management, and verification of surgical tools and other articles within a healthcare facility by both humans and robots alike. Technologies discussed below may therefore establish a hybrid asset tracking network with tracking tags capable of withstanding the rigorous environment of an autoclave while still providing precise location information. Indeed, certain heat-resistant technologies may be used as described below since they can withstand the extreme temperatures associated with the autoclave sterilization process while still transmitting data using wireless signals that can advantageously travel through metallic surfaces and water-rich barriers, making such tracking tags particularly suited for tracking metal surgical tools housed within metallic sterilization trays during and after sterilization. In addition, the hybrid asset tracking system may help ensure the traceability, sterilization status confirmation, and location monitoring of surgical assets to enhance operational efficiency and maintain stringent standards of sterilization.

The system itself may use, among other tracking technologies, tracking tags that transmit wireless signals in frequencies consistent with the IEEE 1902.1 standard. These tags may be affixed to surgical tool trays since they are capable of enduring the severe conditions of autoclave sterilization, including temperatures of at least 250° F. The tags may also include environmental sensors to confirm the integrity of sterilization cycles and to detect moisture indicative of wet load conditions, all without disturbing the integrity of sterile wraps used to wrap the trays post-sterilization (to help keep them sterilized until their next use). However, note that other heat-resistant near-field tracking tags besides those consistent with the IEEE 1902.1 standard may also be used in some implementations.

In either case, other wireless communication tags with longer range can also be used for humans and robots to track other items within the hospital as part of the hybrid asset tracking system. These longer-range tags may include Bluetooth (BT) tags, though here again other types of tracking tags may be used, such as Wi-Fi and/or ultrawideband (UWB) tracking tags. In either case, these tags may be, among other things, affixed to case carts that transport the sterilization trays mentioned above. These tags may thus facilitate the broad-area localization of surgical tools as they are moved across various departments and operating rooms within the hospital using case carts.

In embodiments where BT tags in particular are used, signals from the BT tags may be transmitted in the frequency range of 2.400 GHz to 2.48 GHz consistent with BT communication protocols such as BT 6.0 as conforming to IEEE standard 802.15. And to reiterate, other longer-range tags may also be used, such as Wi-Fi tags for sensing by Wi-Fi receivers according to the IEEE standard 802.11 and also UWB tags for sensing by UWB receivers according to the IEEE standard 802.15.4.

In addition, utilization of two or more distinct frequency bands aids in the differentiation and optimization of the different kinds of tags, with the IEEE 1902.1 tags functioning on low frequency bands and the BT tags operating on ultra-high or super high frequency bands.

Furthermore, the system may use receivers for both kinds of tags that are strategically placed within the hospital to read the tags as they pass by. The data indicated in the signals may then be transmitted by the readers to a network server which may then process the data and register the presence and status of the sterilization trays and other items as they pass specific checkpoints containing one or both kinds of receivers.

Accordingly, hybrid asset tracking system may help streamline the workflow associated with the sterilization and utilization of surgical tools, increasing efficiency and reliability, diminishing the risk of infection, and optimizing the availability of tools when and where they are needed most within the hospital setting.

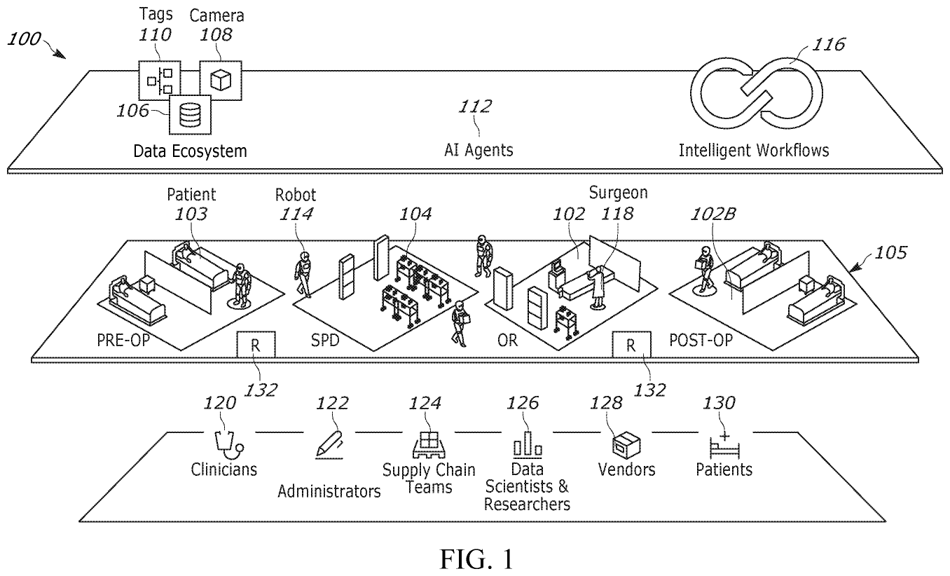

Now in reference to FIG. 1, this figure illustrates a hospital 100 which includes one or more operating rooms (OR) 102 and at least one sterile processing department (SPD) 104 that itself may have an autoclave for sterilizing surgical tools and trays. As illustrated in FIG. 1, pre-operation procedures may include preparation of a patient 103 in a room and may further include including sterilizing the OR and transporting hospital assets to the OR, including trays of sterile surgical instruments (also referred to herein as “tools”) from the SPD 104 to the OR 102. A robot may hand a tool from the tray to a human or robotic surgeon. An operation is then performed in the OR 102 on the patient, after which, as indicated at 102B, post-operation transport and care of the patient 103 to recovery 105 may be performed along with cleanup of the OR and transport of assets back to the appropriate location (such as used tools to the SPD 104, etc.).

In accordance with present principles, to aid in the end-to-end process shown in FIG. 1, a database system 106 stores and correlates data from various sources, including cameras 108 and tracking tags 110 (described more below) along with other sensors including haptic feedback sensors. One or more artificial intelligence (AI) agents 112, also referred to herein as machine learning (ML) models, access the database system 106 and outputs of the cameras 108 and tags 110 to control one or more mobile robots 114, which may be humanoid robots with appropriate end effectors for completing specific manipulative tasks (such as transporting tools within the hospital and assisting in surgeries), according to training of the ML models 112 to implement workflows 116 related to pre-op, surgical, and post-op tasks. The ML models may also be used for asset tracking consistent with present principles. The workflows 116 may be computer-stored in the database system 106 and may evolve over time as one or more ML models iteratively or recursively learn improvements in the workflows.

In this way, the AI-controlled robots 114 can assist in surgeries that may involve one or more humans 118. For example, the robots 114 may assist clinicians 120, administrators 122, supply chain personnel 124, scientists and researchers 126, vendors 128, and patients 130.

One or more of the cameras 108 may be located in the SPD 104 for imaging plural sterilized surgical instruments output by a sterilization assembly such as an autoclave in the SPD. One or more cameras 108 also may be located in the OR 102 to image the operation, including human and robotic activity and medical machines that display data, which may then be classified by, e.g., a machine learning classifier. Tags 110, which may be near field wireless proximity tags as described elsewhere herein, may be attached to hospital assets including tool trays, case carts, gurneys and surgical beds, artificial implants such as prosthetics, etc. and may be tracked by fixed readers 132 to track assets and upload time-stamped location data of the assets to the DB system 106. Further details of the tracking of such assets will now be described.

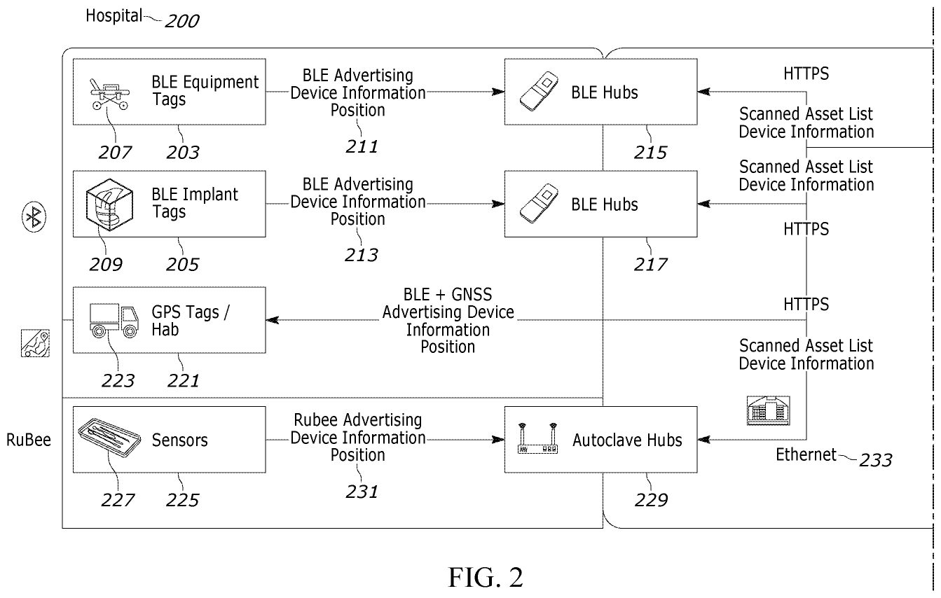

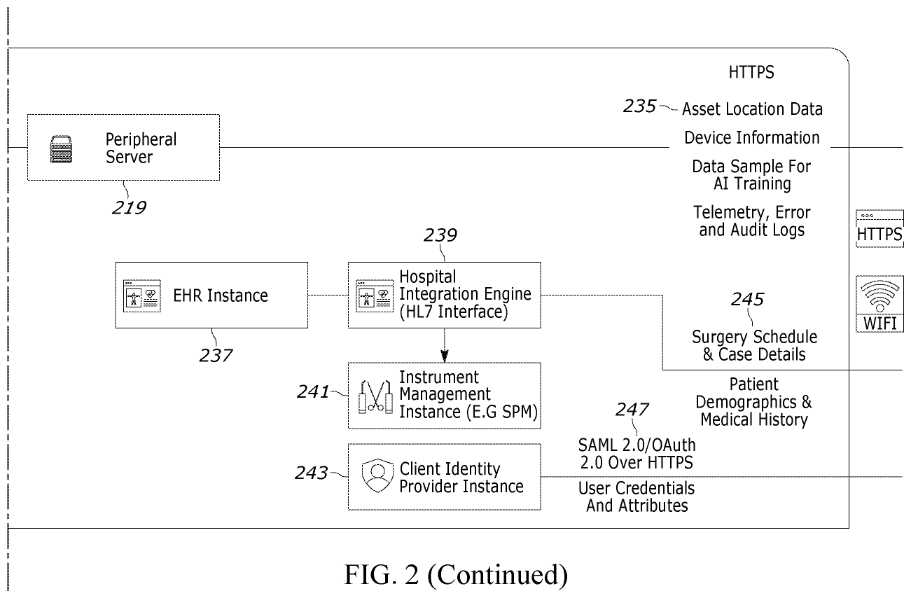

Accordingly, refer now to FIG. 2. This figure shows a schematic diagram of a tracking system consistent with present principles as may be implemented in a healthcare facility such as a hospital 200. As shown, Bluetooth low energy (BLE) or other Bluetooth (BT) equipment tags 203, 205 may be affixed to healthcare-related articles such as a surgical bed 207 and a medical implant 209. The BLE tags 203, 205 may transmit wireless signals 211, 213 to one or more BLE hub devices 215, 217 (more generally, BT tag readers/BT trackers) spaced apart from each other in the hospital 200. The wireless signals 211, 213 may broadcast “advertisement” signals as predefined intervals, such as every five seconds, to indicate their presence. Five seconds is recognized herein as an acceptable time interval that both allows for high-fidelity location tracking and presence monitoring while also minimizing power consumption from a respective battery in each tag 203, 205.

In terms of the advertisement signals themselves, they may include presence signals, device information signals (e.g., respective unique identifiers associated with the respective healthcare article to which the respective tag is attached), and even position information indicating the location of each tag 203, 205 in instances where position receivers are included on the tags 203, 205 (e.g., GPS transceivers). However, it is to be further understood that location of each tag may be determined by the BLE hubs 215, 217 themselves, including through execution of one or more triangulation algorithms and/or one or more received signal strength indicator (RSSI) algorithms as executed at the receiving hub 215/217 and/or a peripheral server 219. Channel sounding algorithms, phased-based ranging algorithms, round trip timing (RTT) algorithms, and/or trilateration algorithms may also be executed to determine the locations of the tags 203, 205.

The server 219 itself may be embodied as a cloud-based device and/or a local device on-site at the hospital. The server 219 may include its own processor system such as a central processing unit (CPU) and/or graphics processing unit (GPU), random access memory (RAM), persistent storage, a network interface, etc. GPUs in particular may be used by the server for artificial intelligence (AI) model execution consistent with this disclosure, though other types of processors on the server 219 may additionally or alternatively be used.

In addition, whether cloud-based or not, the server 219 may include one discrete unit or multiple discrete units acting in coordination. And as shown in FIG. 2 and mentioned above, the hubs 215, 217 may provide device information indicated in the received device information signals to the server 219. The hubs 215, 217 may also provide a respective scanned asset list for all associated healthcare articles for which respective BLE tags have been read by that hub within a predetermined time span (e.g., within the last minute). However, further note that the server 219 may additionally or alternatively generate and maintain the asset list(s) itself and/or maintain a more comprehensive asset list (with up-to-date real-time article locations) based on the device information reported by several of the hubs 215, 217. In either case, further note that the hubs 215, 217 may communicate with the server using a Hypertext Transfer Protocol Secure (HTTPS) protocol, though other communication protocols may be used as well.

FIG. 2 also shows that the mesh network of tracking components shown in this schematic may include global positioning system (GPS) tags and/or a GPS hub 221. For instance, a GPS transceiver (or even a BT reader or combined reader) may be disposed on a vehicle 223 that transports medical implants and other healthcare-related articles to the hospital 200. Accordingly, as the articles are loaded onto the vehicle from a production facility, warehouse, or other off-site location, respective BT tags on each article may be read by the device 221 on the vehicle and their location tracked in real-time using GPS.

In addition, the BT reader and/or GPS hub 221 may transmit, to the server 219 via HTTPS, the advertisement signals (similar to those described above) such as presence signals, device information signals like respective identifiers associated with the respective healthcare article, and position information signals. The device 221 affixed to the vehicle 223 may also transmit global navigation satellite system (GNSS) data indicating the global location of the articles and/or vehicle 223 to the server 219 for real-time location tracking of the respective articles themselves.

Still further, in one particular instance, the hub 221 that goes on the vehicle 223 may have each of a BT transceiver, a GPS/GNS transceiver, and a cellular communications transceiver. This may help make the hub 221 a mobile hub that can detect BT/BLE tags nearby on the vehicle 223 (as well as other types of tags on the vehicle) to then associate the detected tags with a current location of the vehicle as itself determined using the vehicle's GPS/GNS transceiver. The hub 221 may then transmit the respective tag identifiers and GPS-based locations for the tags to the server 219 using a cellular network (or other network).

Still in reference to FIG. 2, other network tracking components may also be implemented consistent with present principles. Those components may include one or more sensor apparatus 225 configured to communicate in a frequency range of 30-900 kHz according to the Institute of Electrical and Electronics Engineers (IEEE) 1902.1 standard/communication protocol. In non-limiting examples, each apparatus 225 may be an autoclave tag such as a RuBee sensor in particular, though other types of near-field technologies may also be used consistent with present principles (e.g., UWB or Wi-Fi transceivers). Further note that each apparatus 225 may be affixed to its own respective healthcare-related article, such as a respective surgical tool tray 227.

As also shown in FIG. 2, the mesh network of tracking components may further include one or more autoclave tag hubs 229 (more generally, autoclave tag readers) that may be configured to read wireless magnetic signals 231 from the apparatus 225 (as, again, communicating in the frequency range of 30-900 kHz). The signals may indicate advertisement signals, device information and even position information similar to the description above. The hub(s) 229, which may have an ethernet connection 233 or other network connection, may then transmit any of the data received via the signals 231 to the server 219 using HTTPS. Also note in addition that the hub(s) 229 may also transmit, to the server 219, a respective scanned asset list for all associated healthcare articles for which respective autoclave tags have been read by that hub within a predetermined time span (e.g., within the last minute). However, further note that the server 219 may additionally or alternatively generate and maintain the asset list(s) and/or a more comprehensive asset list (with up-to-date real-time article location) based on the device information reported by several of the hub(s) 229 (e.g., a combined list including assets individually reported by each of the BT hubs 215, 217).

It may therefore be appreciated that the server 219 may store and maintain a comprehensive, up-to-date database of all articles within the hospital 220 for which a respective tag has been affixed, whether that tag is BT-based, IEEE 1902.1-based, etc. This comprehensive tracking ability allows the server 219 to then execute other functions consistent with present principles, such as setting an operation start time based on the arrival of certain articles to an operating room and presenting a graphical user interface (GUI) on one or more client devices of hospital personnel so that they can view in real-time the location of the various articles as they move to and throughout the hospital 220.

What's more, it is to be understood that certain articles may be affixed with different types of tags depending on how each article will be used. Thus, as autoclave tag 225 can withstand relatively high temperatures without adverse impacts on their hardware or performance, the autoclave tags 225 may be affixed to any articles that will undergo high-heat sterilization in the autoclave of the SPD 104. However, as BT tags 203, 205 have a relatively longer communication range even if they cannot similarly withstand the heat of the autoclave, those types of tags may be more optimal for use to track articles that will not endure the high temperatures of the autoclave, such as the surgical bed 207 and single-use implants 209 (e.g., as already sterilized with the BT tag affixed in the sterile environment).

Still in reference to FIG. 2 and specifically in relation to the server 219, note that the server may transmit to other servers and/or client devices via HTTPS (and/or store itself) various types of data 235 that may be used consistent with present principles. For instance, the data 235 may include asset (article) location data, device information, data samples/training datasets for training of AI models consistent with present principles, telemetry data, and error and audit logs. The data 235 may also be transmitted to various client devices of hospital personnel as needed by those personnel.

In addition, the server 219 and/or any connected device may host an electronic health record (EHR) instance 237 that includes a hospital integration engine 239 (e.g., HL7 interface) and an instrument management instance 241 (e.g., specimen segment (SPM)). The server and/or any connected device may also host a client identity provider instance 243. Note that the EHR instance 237 may, through the hospital integration engine 239 and consistent with applicable privacy protections, transmit surgery schedule and case details as well as patient demographics and medical history data 245 via HTTPS to client devices of hospital personnel, such as surgeons and nurses who might be anticipating performing surgery on the associated patient. And further note that the client identity provider instance 243 may transmit SAML 2.0/OAuth 2.0 data 247 (such as user credentials and attributes) to the client devices for end-user authentication via HTTPS. Also note that these communications may occur over hospital Wi-Fi if desired.

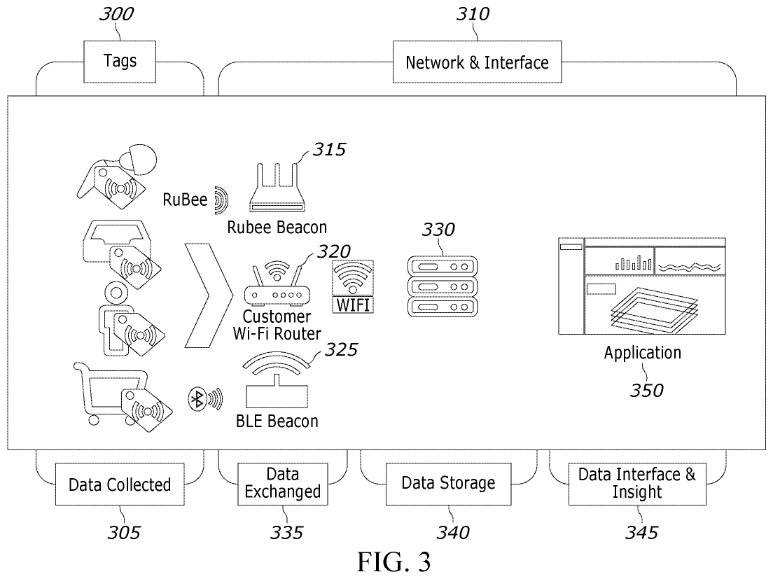

FIG. 3 shows another schematic consistent with present principles. As shown, wireless communication tags 300 such as BT tags and autoclave tags may be affixed to various articles and even hospital personnel for location tracking consistent with present principles so that location and other data may be collected at step 305. Network and interface components 310 are also shown in FIG. 3, including a RuBee beacon 315 (similar to the hub 229 for receiving autoclave tag signals), a Wi-Fi router 320, and a BLE beacon 325 (similar to the hubs 215, 217), all of which may communicate over Wi-Fi and/or other network connections with a server 330 (similar to the server 219). Accordingly, data from the tags 300 may be exchanged at step 335 and then stored at the server 330 at step 340. Then at step 345, a sensor tracking application 350 may be executed to perform functions described herein, including maintaining an up-to-date database of healthcare-related articles as well as tracking their locations.

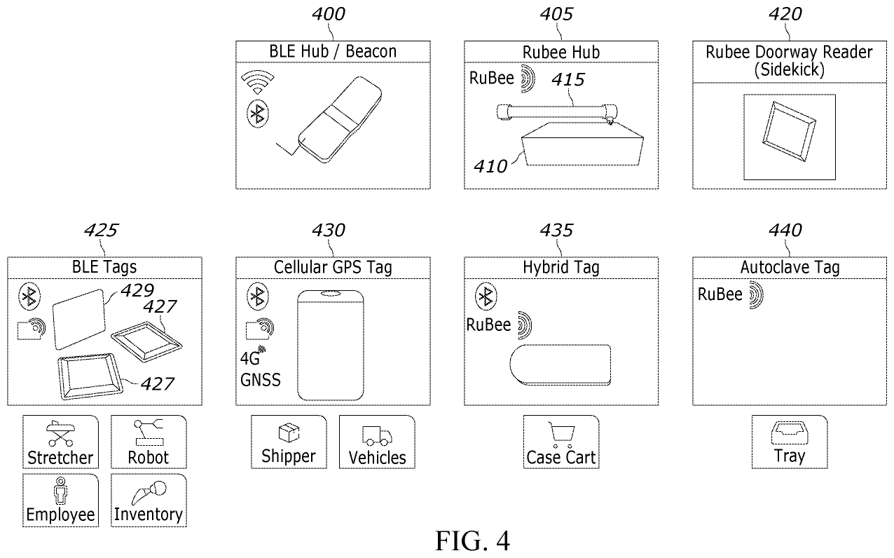

Turning now to FIG. 4, various tracking components that may be implemented consistent with present principles are shown in greater detail. Thus, FIG. 4 shows an example BLE hub/beacon 400 (more generally, BT tracker) that may be configured for BT communication, which may be similar to the hubs 215, 217 discussed above. Thus, the beacon 400 may include one or more BT transceivers to establish a BT reader for zone-based detection of BT tags. In non-limiting examples, the beacon 400 may have a signal range of over three hundred feet, may receive power via a power over ethernet (POE) connection, and may have also have a Wi-Fi transceiver for Wi-Fi communication in addition to its BLE-6 communication capability. The beacon 400 may include other components as well, such as a CPU, RAM, persistent storage, other network transceivers, etc.

FIG. 4 also shows that a RuBee hub 405 with RuBee transceiver may be configured for communication according to the IEEE 1902.1 standard and may be similar to the hub 229 discussed above. The hub 405 itself may include both a RuBee reader 410 (sometimes referred to as a “sidewinder”) and one or more wired RuBee antennas 415 (e.g., four antennas each including a ferrite rod with copper wound around it, for example) for transmitting wireless signals in the frequency range of 30-900 kHz. The hub 405 may also include other components as well, such as a CPU, RAM, persistent storage, other network transceivers, etc.

Each hub 405 might be coupled to a table or storage rack with the hospital, for example. In addition, each hub 405 may establish, in non-limiting embodiments, an integrated server and other circuitry to control the antennas 415, and may also have a network transceiver for ethernet connectivity in particular (e.g., for a POE connection to also power the hub 405). And note that the antennas 415 themselves may each have, in non-limiting embodiments, a four to six foot signal detection range and low radio frequency (RF) pollution capability.

In addition, a RuBee device 420 with a RuBee transceiver may also be used consistent with the description herein (sometimes referred to as a “sidekick”). The device 420 may be positioned adjacent to a doorway within a healthcare facility (e.g., mounted to a wall next to the doorway) to read an autoclave tag as it passes by (as affixed to an article for the device 420 to thus report the current location of the article as being at or near the doorway itself). The device 420 may also have integrated RuBee antennas (e.g., with a two to four foot range) and readers, as will be described in greater detail later. The device 420 may also be, in non-limiting embodiments, directly wired to a central processing unit (CPU) via a POE connection that also powers the device 420. Additionally or alternatively, the device 420 itself may include hardware components such as a CPU, RAM, persistent storage, other network transceivers, etc.

FIG. 4 further shows that one or more BT tags 425, such as Bluetooth low energy (BLE) tags in particular, may be implemented consistent with present principles. Each tag 425 may include its own respective BT transceiver (e.g., and other hardware components such as a CPU or other IC, persistent storage, etc.). The tags 425 may include, for example, rectangular parallelepiped BT tags 427 and more-flat BLE sticker tags 429. Also note that as indicated in FIG. 4, other types of radio frequency identification (RFID) tags and communication standards may be used in addition to or in lieu of BT. Furthermore, other types of tags and communication standards beyond RFID may also be used.

For example, Wi-Fi and/or UWB tags may be used for location tracking of various articles within a healthcare facility. In various examples, UWB communication may involve use of low energy, short-range, high-bandwidth pulse communication over a relatively large portion of the radio spectrum. Thus, for example, an ultra-wideband signal/pulse may be established by a radio signal with fractional bandwidth greater than 20% and/or a bandwidth greater than 500 MHz, with communication occurring by using multiple frequencies (e.g., concurrently) in the frequency range from 3.1 to 10.6 GHz. Also for UWB communication, various UWB algorithms may be used to determine the location of a UWB tag, such as time difference of arrival (TDoA) algorithms and/or angle of arrival (AoA) algorithms.

In any case, note that in non-limiting embodiments the tags 425 may broadcast advertisements at intervals of every five seconds via whatever wireless communication type is being used.

Additionally, for the BT tags 427 in particular, the tags 427 may include a replaceable battery with a three to five year battery life. For the BT sticker tags 429, they main be RFID-scannable with a battery of one to two years of life, making them more disposable. What's more, note that FIG. 4 shows in relation to the BT tags 425 that they may be affixed to various healthcare-related articles that will not be exposed to the high temperatures of the autoclave, such as but not limited to stretchers/gurneys, surgical beds, robots, case carts, hospital personnel, and even already-sterilized single-use inventory such as patient implants.

As also shown in FIG. 4, cellular GPS tags 430 may also be used as part of the hybrid asset tracking system, such as for vehicle tracking as set forth above. But note that the tag 430 might also include not just a GPS transceiver (e.g., for GNSS communication), but also BT and other RFID transceivers as well. The tag(s) 430 may also include wireless cellular transceivers such as 4G, 5G, and/or 6G cellular transceivers (e.g., for communication and location tracking over a wireless cellular network). FIG. 4 further demonstrates that the tags 430 may be affixed to shipping containers and vehicles, as well as other articles.

Still in reference to FIG. 4, hybrid tags 435 may also be used for certain healthcare-related items, such as case carts that transport, organize, and store trays with surgical instruments, tools, and other supplies between the hospital's SPD and an operating room (OR). Being a hybrid tag, each tag 435 may include both a BT transceiver and a RuBee transceiver for redundancy and high-fidelity location tracking. The tags 435 may also have their own built-in batteries that may have a three-year battery life, for example.

What's more, autoclave tags 440 similar to the apparatus 225 that may also be used as part of the tracking system, where each autoclave tag may include its own RuBee transceiver as well as other heat-resistant components for affixation to a surgical tool tray so that each tag 440 may be autoclavable to withstand the heat of the autoclave itself. Also note that the tags 440 may include other components described herein, such as a processor or other integrated circuit (IC) as well as RAM, storage, etc. on a printed circuit board (PCB) to log temperature readings from a temperature sensor in the tag 400 and humidity readings from a humidity sensor in the tag 400 (e.g., including an overall history of both temperature and humidity readings for that particular autoclave tag 440). Each tag 440 may also include its own battery with, for example, over three years of battery life.

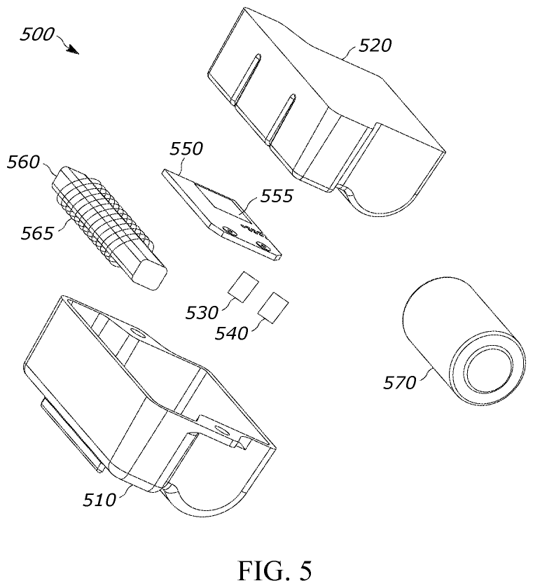

Now in reference to FIG. 5, components of an example autoclave tag 500 (similar to the tag 440) are shown in exploded view. The tag 500 may include a rigid autoclave housing radel 510 (more generally, a housing). The housing 510 may be made of a polyphenylsulfone (PPSU) in non-limiting embodiments to be compliant with ISO 10993 biocompatibility.

The tag 500 may also include a potting 520 that goes within the housing 510. The potting 520 may be established by a flexible epoxy with a cyanoacrylate (CA) adhesive, for example. However, other types of epoxy and other types of potting material may also be used to also protect the components inside the potting 520 from sudden temperature changes that might otherwise be experienced during the autoclave sterilization process while also providing additional mechanical protection for those components during the autoclave process. Accordingly, other examples of potting material that may be used in addition to or in lieu of epoxy include polyurethanes and silicones.

The tag 500 may further include environmental sensors, such as a humidity sensor 530 coupled to the housing 510 and disposed at least partially within the potting 520. Another example environmental sensor that might be included in the tag 500 is a temperature sensor 540 that may also be coupled to the housing and disposed at least partially within the potting. And in non-limiting examples, both of the sensors 530, 540 may be embodied on or otherwise electrically coupled to a printed circuit bord (PCB) 550 of the tag 500 along with other components mentioned above.

Thus, the PCB 550 itself may be coupled to the housing and disposed at least partially within the potting. The PCB 550 may include one or more portions of a wireless communication assembly of the tag 500. As such, the PCB 550 may support an integrated circuit (IC) 555 communicatively coupled to a ferrite rod antenna 560 with copper wire 565 wound therearound, with the antenna 560 also forming part of the wireless communication assembly. In certain example embodiments, the ferrite rod can be cylindrical or an extruded rectangle, though other shapes may also be implemented. Further note that other suitable antennas may also be used beyond ferrite rod antennas wrapped in copper wire.

The IC 555 may include/establish one or more microprocessors or other types of processors such as a CPU, with the PCB 550 also supporting other computer-related components such as RAM, persistent storage, and a Rubee transceiver. Thus, the IC 555 on the PCB 550 may be configured to use the antenna 560 and transceiver to transmit wireless signals in a frequency range of 30-900 kHz. In one particular non-limiting embodiment, the IC 555 may be configured to use the antenna 560 to transmit wireless signals in the frequency range of 130-132 kHz for data transmission and, more particularly, in the frequency of 131 kHz for transmission of the wireless data signals per to the IEEE standard 1902.1.

As for the wireless signals themselves that are transmitted in the frequency range of 30-900 kHz, they may indicate a variety of different things consistent with the disclosure herein. Accordingly, in one particular example, the tag 500 may transmit one or more first wireless signals indicating a unique identifier for a surgical tool tray associated with the tag 500, one or more second wireless signals indicating humidity data generated based on input from the humidity sensor 530, and one or more third wireless signals indicating temperature data generated based on input from the temperature sensor 540. And note here that the persistent storage and/or RAM on the PCB 550 may store the identifier, the humidity data, and the temperature data. The humidity data and temperature data in particular may be stored responsive to the IC 555 receiving corresponding readings from the respective sensors 530, 540 themselves. The IC 555 may then access the identifier and environmental data (e.g., temperature and humidity data) as stored in local storage to use the wireless transceiver on the PCB 550 to subsequently generate the first, second, and third wireless signals mentioned above using the identifier and environmental data as stored in the local storage.

As also shown in FIG. 5, the tag 500 may include a high temperature-resistant battery 570 disposed within the housing 510 to provide power to the wireless communication assembly, PCB 550, IC 555, and sensors 530, 540. The battery 570 may therefore be configured to withstand relatively high temperatures, including those the tag 500 might endure during sterilization in the autoclave, and as such may be a Tadiran Lithium High-Temperature (TLH) 1/2 AA battery in non-limiting examples.

Also note that in some examples, the PCB 550 and other electronic components connected to it as described above may be powered down according to commands from the IC 555 based on a temperature reading from the sensor 540 rising to 85 degrees Celsius (C.) (e.g., plus or minus five degrees C.) for auto-protected sleep at high temperatures endured during the sterilization process to protect those electronic components. Then responsive to going back below that threshold, the components may be powered back on.

In addition, though not shown for simplicity, in some embodiments the PCB 550 may support an accelerometer, inclinometer, magnetometer, and/or a gyroscope to give positional information on the tag 500 (which may then be transmitted to readers throughout the hospital), as well as a BT/BLE transceiver and/or GPS transceiver to enable tracking of the tag 500 within and outside a hospital using BT and GPS consistent with present principles.

Also in some examples, the tag 500 may be wired or otherwise in communication with an antenna built into the associated instrument tray itself to enable the transceiving of signals reaching through the tray fenestrations. Another antenna also in communication with the tag 500 may be incorporated into the blue wrap of the tray and/or tape that seals the blue wrap to keep the items in side sterile for additional enhanced transceiving capability. The antenna and/or tracking sensor(s) can also be incorporated into the orange clip that is utilized to confirm the lid to the tray is sealed, also for enhanced transceiving capability.

Referring back to the potting 520, note that while the sensors 530, 540 may be encased in the potting 520 along with the PCB 550, IC 555, antenna 560, and battery 570 to protect those components from sudden temperature changes due to the heat of the autoclave sterilization process, the potting 520 may still be porous for the humidity sensor 530 to sense humidity external to the housing 510 and for the temperature sensor 540 to sense temperature external to the housing 510, such as in instances where the potting 520 includes epoxy for example.

Additionally or alternatively, the potting 520 may be configured with one or more man-made access holes or other openings (e.g., larger than the epoxy's pores) for the humidity sensor 530 to still sense humidity external to the housing 510 and for the temperature sensor 540 to still sense temperature external to the housing 510. The one or more man-made openings may be bored or drilled into the potting 520 after the potting material has cured, for example. Or the potting material may be poured with an opening maintained via a mold that is used during the material pouring process, pre-curing. Regardless, the man-made openings may be different from any pores of the potting 520 that form in the epoxy or other material of the potting 520 based on the material itself curing. But whether pores or man-made openings are used, it is to be understood that these open areas within the potting 510 allow the sensors 530, 540 to still sense environmental conditions external to the tag 500 while the potting 510 itself nonetheless slows down temperature changes experienced by those components due autoclave heat that might otherwise accelerate degradation of the sensors 530, 540.

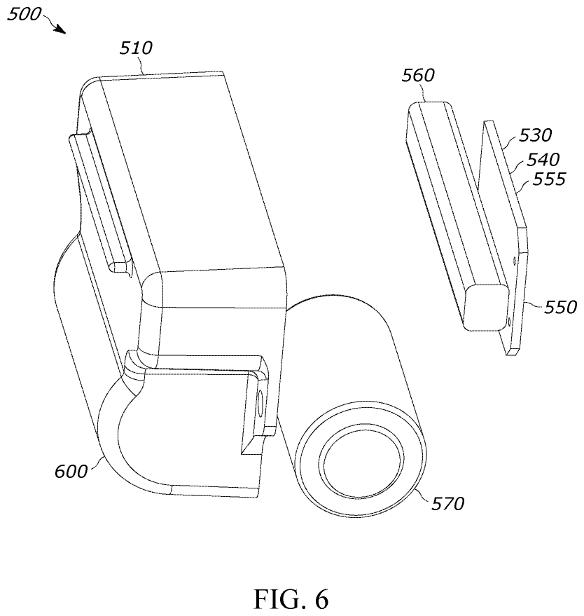

Turning now to FIG. 6, this figure shows a perspective view demonstrating assembly of the tag 500 once the potting 520 has been shape-fitted within the housing 510 itself. As shown, a partially cylindrical potion 600 of the housing 510 (and potting 520) may receive the battery 570, while a rectangular prism-shaped portion of the housing 510 may receive the antenna 560 and PCB 550. Further note that the environmental sensors 530, 540 and IC 555 have been physically and electrically coupled to one side of the IC 550 as shown.

Once the internal electronic components are fitted within the tag 500, an additional potting layer may be positioned over the open side of the housing 510 and then a door or side (that may also establish part of the housing 510) may be secured in place to fully enclose the electronic components within the potting and tag housing.

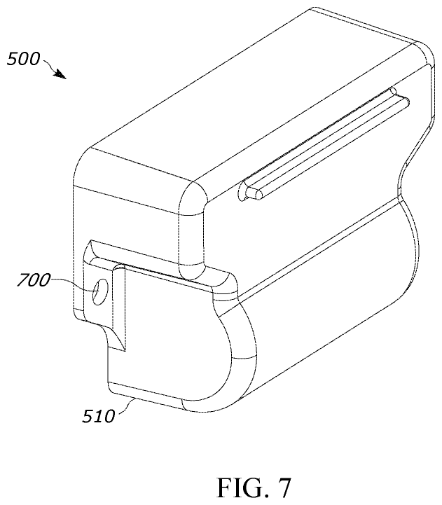

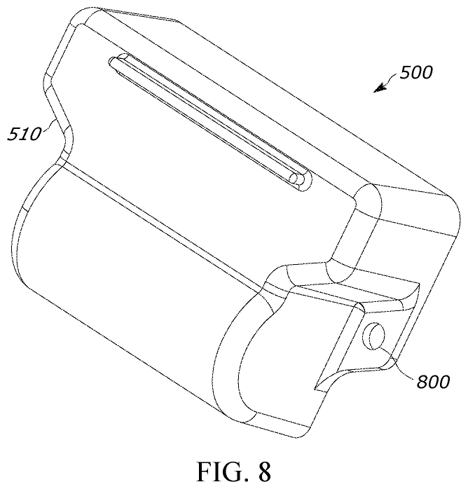

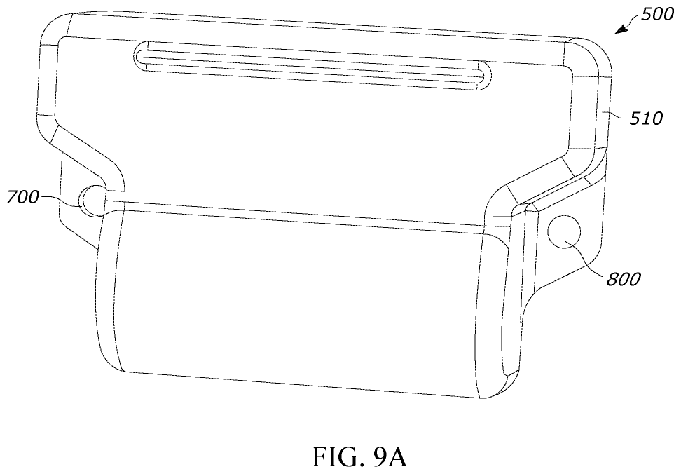

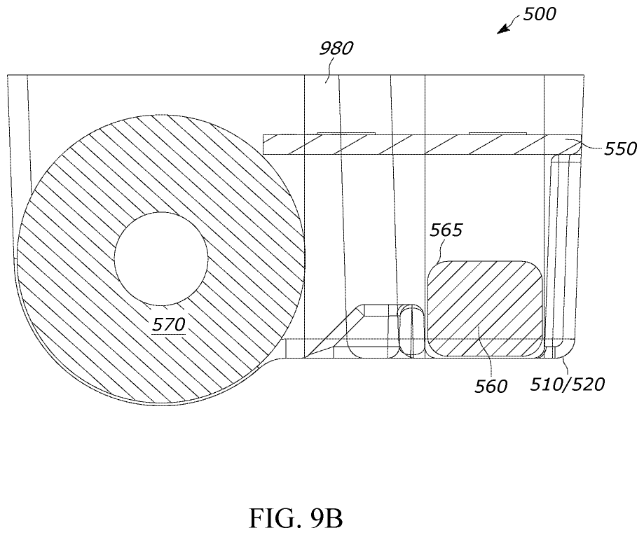

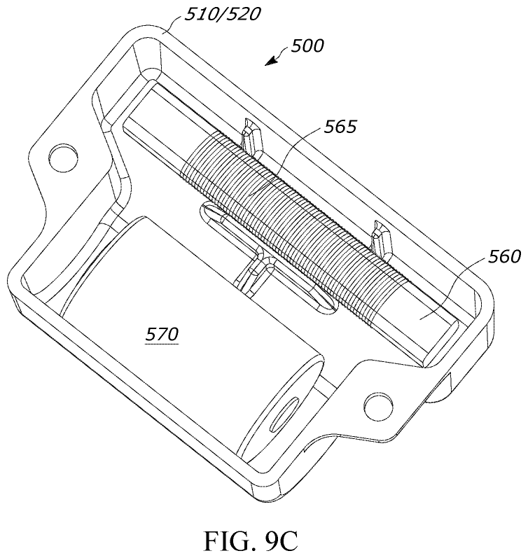

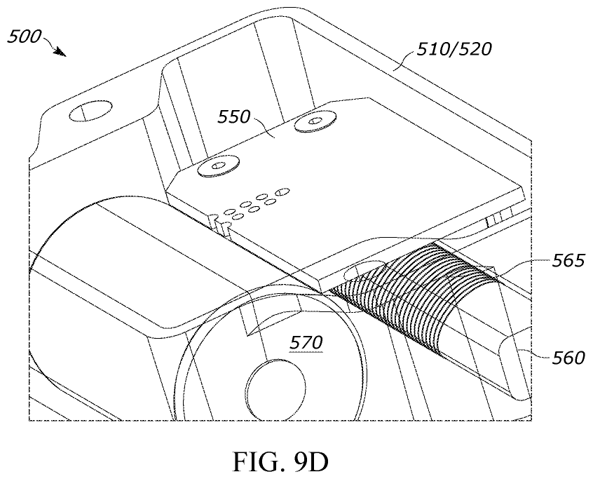

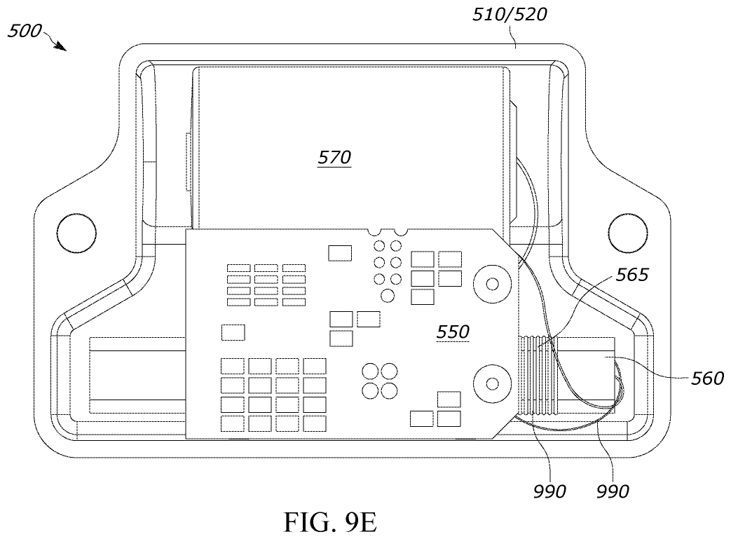

Now in reference to FIGS. 7 and 8, these figures show perspective views of the tag 500 as fully assembled. In addition, FIG. 7 shows that the housing 510 may include a left-side opening 700, while FIG. 8 shows that the housing 510 may include a right-side opening 800. Both of the openings 700, 800 may extend in a plane that is parallel to the plane of the back face of the housing 510 establishing the door or panel that encloses the electronic components as described above. FIG. 9A then shows an additional perspective view in which both openings 700, 800 are shown. The openings 700, 800 may then be engaged with opposing ends of a stainless steel wire or other connector that also wraps around part of a surgical tool tray to secure the tag 500 to the tray (e.g., stainless steel being used for resiliency despite multiple sterilizations).

Turning to FIG. 9B, a side cutaway view of the autoclave tag 500 is shown to further demonstrate the components of the tag 500 in relation to each other when the tag 500 is fully assembled per one example embodiment. Note here that in some instances, the combined potting/door 980 as mentioned above may have a thickness of 2 millimeters/0.0787 inches.

FIGS. 9C and 9D then show other cutaway views of the tag 500, while FIG. 9E shows yet another cutaway view demonstrating that the battery 570 may be wired to the antenna 560 and board 550 via one or more wires 990 to provide power to those components.

The tags 500 may be coupled to a surgical tool tray for tracking the tray, and hence tools within the tray, through the autoclave process as well as throughout other portions of the hospital or other healthcare facility. However, further note that the tags 500 may be used for other purposes as well. For example, the tags 500 may be used to monitor sterilization of the associated tray itself.

Thus, in one non-limiting example, the IC 555 may be configured to store, in the storage embodied on PCB 550, an indication of how many times a temperature sensed by the temperature sensor 540 rose to a predetermined temperature and how long it stayed above that temperature before going back below that temperature. The predetermined temperature may be 121° C., which is considered an acceptable temperature for tray and tool sterilization should the temperature stay at or above 121° C. for thirty to sixty minutes. Thus, in some examples the IC 555 may track and store not just how many times the temperature rose to the predetermined threshold, but, for each time, how long the sensed temperature stayed at or above that threshold continuously (without dipping below the threshold at any point) to thus confirm a proper sterilization procedure was completed. That procedure may then be logged in the tag's storage along with a timestamp for when the sterilization occurred and how long the tag's temperature persisted above that threshold so that all that data may then be sent to an autoclave tag reader when within range. In addition, along with the sterilization count-how many times the temperature rose to the predetermined temperature and stayed above the predetermined temperature for a threshold amount of time-the IC 555 may also be configured to store, in the local storage of the tag 500, an indication of whether the tray is in fact sterilized based on that criteria and a total sterilization count, which may also be transmitted to the autoclave tag reader when within range.

Refer now to FIG. 10. This figure shows a perspective view of an autoclave tag 500 as coupled to a surgical tool tray 1000. Note that the tray 1000 may contain plural surgical tools such as surgical scissors 1010 and surgical thumb forceps 1020. Also note that the tools are placed on top of sterile paper or a sterile mat 1030, which itself may be placed on the bottom of the tray 1000. Further note that the tray 1000 itself may include perforations, some of which may be used to attach the tag 500 to a vertical sidewall of the tray 1000 using a stainless steel wire or other connector as mentioned above.

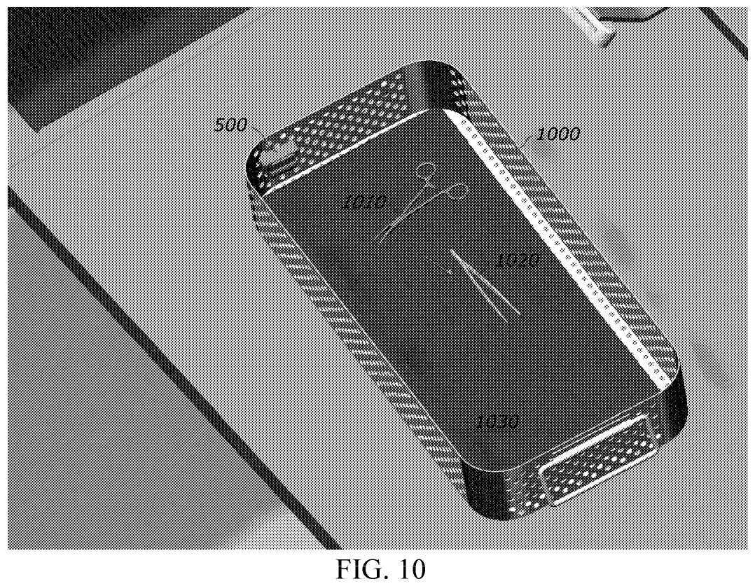

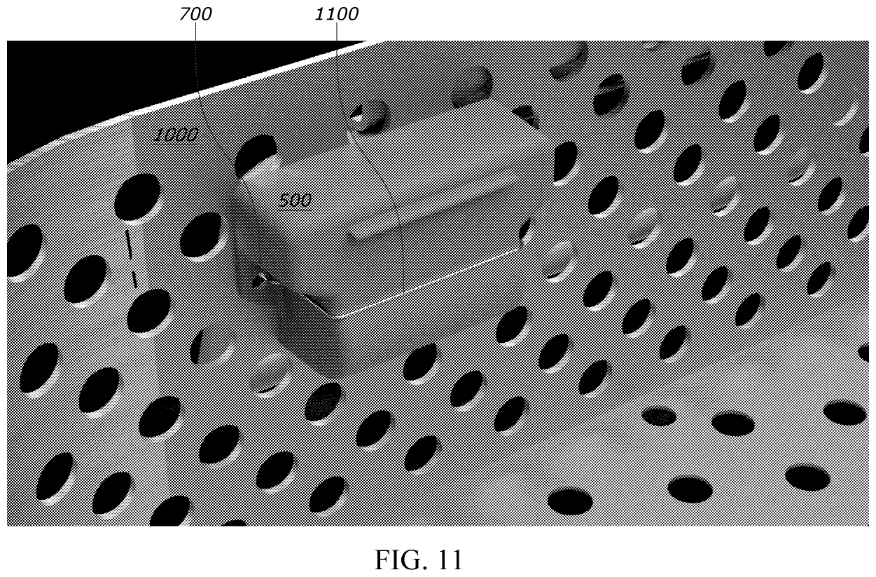

FIG. 11 then shows a close-up perspective view of the tag 500 as coupled to the tray 1000 at an inner face of the sidewall, with a wire 1100 shown as coupling the tag 500 to the tray through the openings 700, 800. FIG. 12 shows the outer face of the tray sidewall to demonstrate that the wire 1100 (or metal bar or other connector) may extend through first and second perforations 1210, 1220 to couple the tag 500 to the structure of the tray 1000 once the wire or bar 1100 has been extended around the tag 500 and through the openings 700, 800. Further note that other coupling mechanisms may be used to secure the tag 500 to the tray 1000. Also in some non-limiting examples, the wire or bar 1100 may be made integral with the tag 500 itself (e.g., where both the tray 1000 and tag 500 are manufactured together). In either case, as mentioned above the tag 500 may be affixed to the tray 1000 for location tracking of the tray as well as performance of other functions throughout the tray's travel within the hospital, including during sterilization in the autoclave.

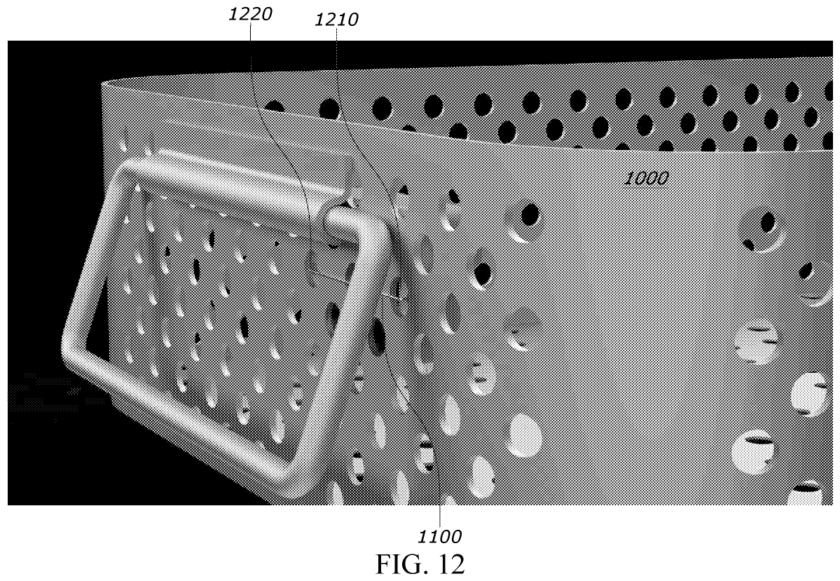

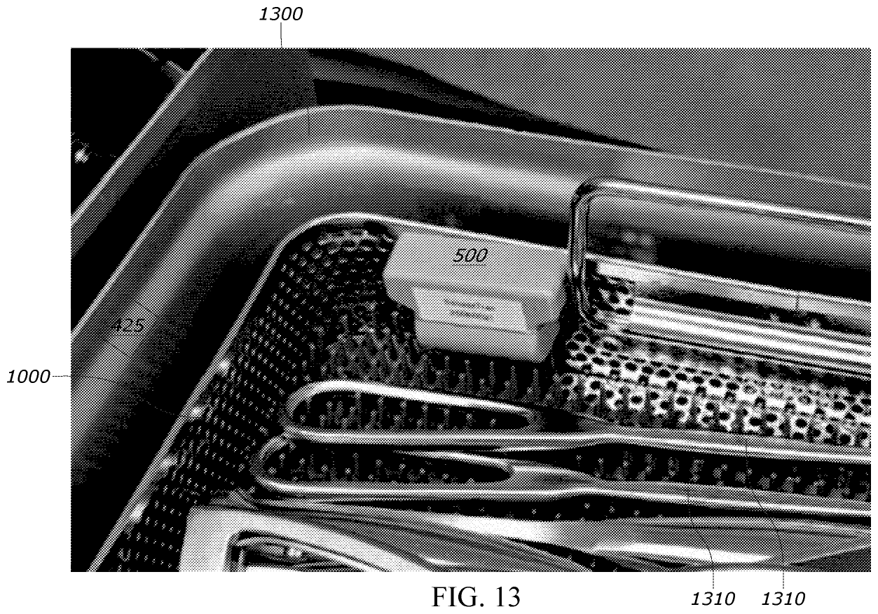

Turning to FIG. 13, this figure shows a partial perspective view of the tray 1000 with tag 500 secured to it with the tray 1000 itself placed on a case cart 1300. Also note that plural surgical tools 1310 are shown within the tray 1000. Further note that one or more BT tags 425 may be affixed to the case cart 1300 itself for independent location tracking of the cart. It may therefore be appreciated based on this figure that multiple tracking modalities (e.g., IEEE 1902.1 and BT) may be used to track the tray and cart as they move about the hospital.

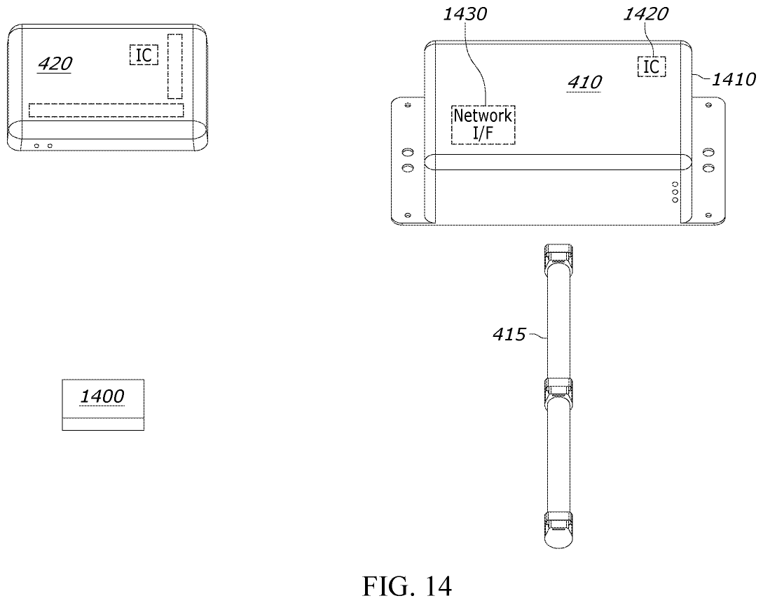

Now in reference to FIG. 14, this figure shows various different kinds of autoclave tag readers that may be used consistent with present principles, including an autoclave tag hub 405 with autoclave tag reader 410 and autoclave tag reader antenna 415. FIG. 14 also shows an autoclave tag doorway reader 420. Several of these devices 405, 420 may be placed throughout a hospital to establish a mesh of autoclave tag readers for tracking of autoclave as they are positioned in and move about the hospital.

Accordingly, the hub 405 may be positioned on a storage shelf or rack to monitor (via a respective autoclave tag 500) surgical tool trays that have been sterilized and stocked for subsequent surgeries. The doorway reader 420 may be positioned adjacent to a doorway within the healthcare facility, such as the doorway to an operating room, and powered over a POE connection so that surgical tool tray can be read via its respective tag 500 as entering the operating room. An additional doorway read 420 may be positioned adjacent to a doorway out of the SPD to read a tool tray's autoclave tag as it exists the SPD.

FIG. 14 further shows that a BT reader 1400, which may also be mounted adjacent to one or more of the same doorways to read any BT tag attached to a case cart that is carrying the tray itself. Accordingly, with both of the readers 420, 1400 reading their respective tags within a threshold time of each other (e.g., within ten seconds), the network (e.g., server 219) may infer that a particular tray as identified via the unique identifier for its respective autoclave tag 500 is being transported through the associated doorway by a particular case cart also identified via the unique identifier for its respective BT tag 425 as read around the same time. Additionally or alternatively, a hybrid tag 435 with both an autoclave tag and BT tag incorporated into the same discrete device may be attached to the case cart so that both of those tags may be read around the same time for increased confidence as the autoclave tag on the hybrid tag 435 and the autoclave tag 500 on the tray itself would be read simultaneously or within a very short threshold time of each other (e.g., one second), with the BT tag in the hybrid tag 435 also being tracked to the same location at the same time.

Still in reference to FIG. 14 and now describing the components of the hub 405 in greater detail, it is to be understood that the hub 405 may include a rigid housing 1410. The hub 405 may also include a wireless communication assembly coupled to the housing 1410. The wireless communication assembly may include both the antenna 415 (e.g., cylindrical or rectangular ferrite rod antenna with copper wire wrapped around it, or other suitable antenna) as well as an integrated circuit (IC) 1420 communicatively coupled to the antenna 415 (e.g., wired connection). The IC 1420 may be configured to use the antenna 415 to receive wireless signals in a frequency range of 30-900 kHz. Thus, in one particular example, the IC 1420 may be configured to receive, in the frequency range of 30-900 kHz, each of one or more first wireless signals, one or more second wireless signals, and/or one or more third wireless signals. The one or more first wireless signals may indicate an identifier for a surgical tool tray associated with another apparatus different from hub 405, such as an autoclave tag 440 and/or autoclave tag 500. The one or more second wireless signals may indicate humidity data generated based on input from a humidity sensor on the respective autoclave tag. And the one or more third wireless signals may indicate temperature data generated based on input from a temperature sensor on the respective autoclave tag.

Note that in various examples, the IC 1420 may be embodied as a microprocessor, a digital signal processor (DSP), a central processing unit (CPU), a graphics processing unit (GPU), and/or other processor system of the hub 405. Also in various examples, the IC 1420 may be included on a PCB within the hub 405, such as a motherboard. The PCB 550 may also be connected to other computer-related components such as RAM, persistent storage, and a RuBee transceiver. Thus, while the IC 1420 may be configured to use the antenna 415 and transceiver to receive wireless signals in a frequency range of 30-900 kHz, in one particular non-limiting embodiment, the IC 1420 may be configured to use the antenna 415 to receive wireless signals in the frequency range of 130-132 kHz as used for data transmission according to the IEEE 1902.1 standard. And if desired, the frequency of 131 kHz in particular may be used, including for receipt of the wireless signals according to the IEEE standard 1902.1.

Further note that in various examples, the hub 405 may establish a server that includes one or more network transceivers/network interfaces 1430 to communicate over a network such as the Internet or a secure healthcare facility local area network (e.g., using HTTPS communication). In one particular implementation, the network transceiver include an ethernet port for both ethernet communication and POE power supply to the hub 405. And as mentioned above, since the IC 1420 may establish a processor system and since the PCB on which the IC 1420 is disposed may further include persistent storage, the storage may store instructions executable by that processor system to associate the identifier from the tag 500 indicated via the one or more first wireless signals (e.g., as associated with an attached surgical tool tray) with a current (known) location of the hub 405 itself. These instructions may also be executable to transmit an indication to another network node, such as the server 219, of the current location as being associated with the identifier.

What's more, in some examples the hub 405 may track all the tags 500 it senses and, in such an instance, execute instructions to store and then transmit, to the other network node(s), a list of healthcare articles that have been determined by the hub 405 as being proximate to a current location of the hub 405. The list may indicate the respective surgical tool trays and/or other articles associated with respective identifiers for respective tags 500 that have been sensed. Also note that different lists may be transmitted at different times to keep the system up-to-date with what articles are currently sensed by the hub 405 and therefore are currently proximate to the hub 405 at the hub's known location. Therefore, the hub 405 may transmit, to the network node at a first time, a first list of healthcare articles that have been determined by the hub 405 as being proximate to the current location of the hub 405 during a first time span based on wireless signals received from the tags 500 during the first time span (e.g., one minute or five minutes).

Then during a second time span later than the first time span, the hub 405 may receive additional wireless signals in the frequency range of 30-900 kHz indicating respective identifiers for respective surgical tool trays and other articles associated with different tags 500 that are sensed as proximate to the hub 405 during the second time span. The hub 405 may then transmit, to the network node, a second list of healthcare articles that have been determined by the hub 405 after the first time span as being proximate to the current location of the hub 405, with the second list indicating the respective surgical tool trays or other articles associated with the respective identifier sensed during the second time span. Further still, note that if the hub 405 determines at or later than the first time that no wireless signals have been received at the hub 405 for at least a threshold amount of time from certain tags 405 that were sensed during the first time span, the hub 405 may decline to include an indication that the associated surgical tool trays are proximate to the current location of the hub 405 in the second list that is sent. Thus, each list may only include identifiers for trays or other articles associated with various tags that have been sensed during the most-recent time span.

In addition, note that the hub 405 may transmit still other data to the other network node(s) like the sever 219 based on data indicated in the wireless signals received from any autoclave tags 500 read by the hub 405. For instance, the hub 405 may transmit, to the network node, an indication regarding whether a certain apparatus associated with a given tag 500 that is sensed includes a wet load of surgical tools. The indication may be generated based on humidity data received from the respective tag 500 itself as received from the respective humidity sensor on the respective tag 500. And further note that a wet load may be inferred when the humidity data indicates sensed humidity above a threshold level (e.g., sixty percent humidity).

Likewise, the hub 405 may transmit, to the network node, an indication regarding whether a certain apparatus associated with a given tag 500 that is sensed has been sterilized. The indication may be generated based on the temperature data received from the respective tag 500 itself as received from the respective temperature sensor on the respective tag 500. Note that the indication itself may have been generated by the tag and/or hub 405 based on the data from the tag 500 that indicates that the threshold temperature mentioned above has been reached for the threshold time (e.g., temperature constantly above 121° C. for at least thirty minutes).

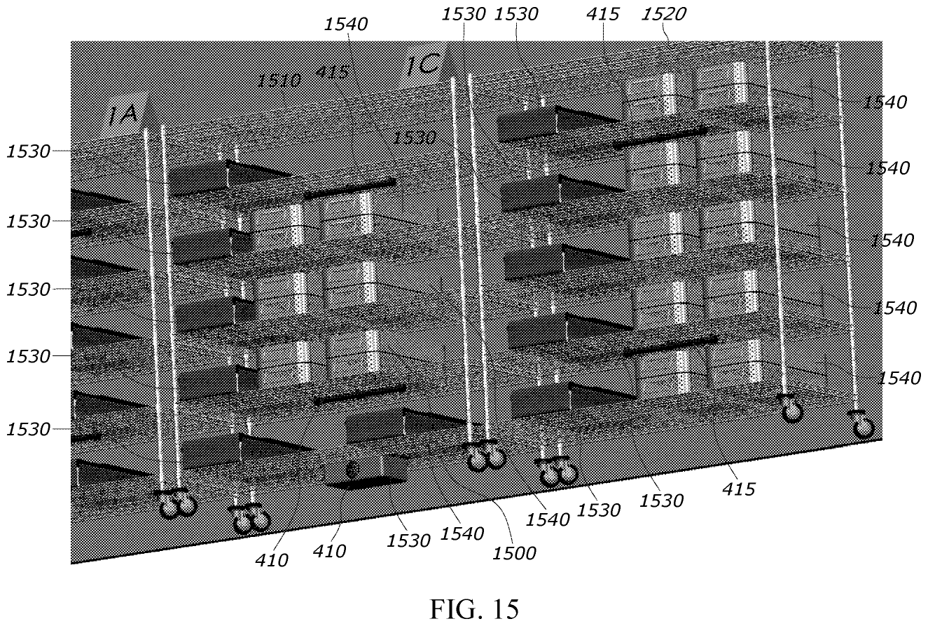

Refer now to FIG. 15. This figure shows an example perspective view of an autoclave tag reader 410 coupled to a ground-facing side of a lower shelf 1500 of a first storage rack 1510 as located the sterile SPD 104 or another sterile location within a healthcare facility. As mentioned above, the reader 410 may be powered by a POE connection or other suitable power connection. Further note that a second storage rack 1520 with its own shelves is also shown, and that both blue-wrapped surgical tool trays 1530 and empty surgical tool trays 1540 are placed on different shelves of each rack 1510, 1520.

As also shown in FIG. 15, plural antennas 415 are shown, each of which may be wired to the reader 410 to establish a respective hub 405. Note that each antenna is coupled to the bottom face of a different shelf to sense respective autoclave tags 500 (not shown) coupled to respective trays 1530, 1540. Note that an antenna 415 need not be placed on every single shelf to sense tags 500 proximate to it in light of its sensing range of four to six feed in non-limiting embodiments. Likewise, a respective reader 410 need not be coupled to each rack and that, in the present instance, a single reader 410 on the rack 1510 may be used to monitor autoclave tags on both of the racks 1510, 1520. But advantageously, the antennas 415 may still receive wireless signals from the tags 500 on the trays 1530, 1540 on each rack for the IC 1420 on the reader 410 to process the data indicated in those signals consistent with the disclosure herein.

Further note that to optimize the wireless signal sensing range of the antennas 415, the antennas 415 may be coupled to a respective shelf horizontally as shown so that the sensing axis of the antennas 415 align with the respective antennas as also arranged horizontally on the respective tags 500 on each tray 1530, 1540. In this way, wireless signals indicating identifiers for each tray 1530, 1540 as well as wireless signals indicating temperature and humidity data for each tray may be communicated from the antennas in the tags 500 to one or more of the antennas 415 with an optimized signal range.

Continuing the detailed description in reference to FIG. 16 but still in reference to the same example as FIG. 15, note that a case cart 1600 now supports plural surgical tool trays 1530, 1540 that were taken off the storage racks 1510/1520. Assume the case cart 1600 is being pushed by a perioperative robot or human through a doorway 1620 from the SPD 104 into a hospital hallway 1640 so that it may eventually be taken to an operating room. What's more, note that an autoclave tag sidekick device 420 has been mounted to an inside wall of the SPD 104 adjacent to the doorway 1620, with it being powered by a POE connection/ethernet wire 1660.

Accordingly, as the cart 1600 passes within four to six feet of the device 420, magnetic signals emitted by the respective tags 500 on each tray 1530, 1540 may be received by the device 420. Signals from each tag 500 may indicate a respective identifier for the associated tray 1530, 1540 to which that tag 500 is attached and, as such, the device 420 may use the identifiers to log with the system that the respective trays have been taken from the SPD 104 and are in transit within the hospital. Other sidekick devices 420 mounted elsewhere in the hospital may then continue to track those trays as they pass by those other sidekick devices 420 as the cart 1600 proceeds to the OR so that the system continues to receive real-time location updates on the location of the trays 1530, 1540 for monitoring and surgery schedule adjustments as warranted. And note that for reasons like redundancy and increase confidence in location tracking, a BT tag 425 and/or hybrid tag 435 affixed to the cart 1600 may be tracked via BT trackers 215, 217 (and device 420 for the RuBee emitter of the tag 435) as those readers are also located throughout the hospital so that the location of the cart 1600 itself can be tracked.

As indicated above, this may be done so that, with the location of the cart 1600 being tracked, the cart 1600 itself may be inferred by the system as carrying certain trays located at the same or similar locations at the same time as the cart 1600 (as themselves sensed via autoclave readers that detect wireless signals from the respective autoclave tags on the trays 1530, 1540) as all those articles move through the hospital together. It may therefore be appreciated that the multiple wireless sensing modalities that are used help create a robust tracking system for carts and trays as they move about in the hospital.

Further note that since signals from each tag 500 may also indicate whether the associated tray has been sterilized and whether the associated tray contains a wet load, once the device 420 receives those signals it may log with the system whether each tray that has been taken from the SPD 104 is in fact sterile and contains a dry load (e.g., transmit indications of those things to the sever 219). If either condition is not met for a respective tray (not sterile or not a dry load of tools), the system may then notify any perioperative robots and/or hospital personnel that are scheduled to help perform an assigned surgery with the compromised tools from that tray so that they are not in fact used. For hospital personnel in particular, for example, a graphical user interface (GUI) may be presented at their client device's display indicating as much. Audible notifications may also be presented using the speakers of the client device's speaker(s). In either case, the notification might indicate something like “Do not use the tools in tray number 42, they are not sterile and/or are still wet.”

For perioperative robots, once the notification is received by the robot, the robot itself may use its own on-bard display and/or speaker to present a similar notification to any nearby human personnel, such as a surgeon near the robot. In this way, the surgeon can be notified that the unsterile or wet-load tools should not be used even if the surgeon's own personal device is not nearby.

In addition, since the system has logged that one or more trays contain tools in an unsterile tray or tools, and/or that one or more trays contain a wet load (damp tools that also might not be sterile since they're damp), still other actions may be taken as well. For instance, the system can send a control command to a robot operating within the hospital to immediately fetch a replacement tray from the SPD 104 and bring that replacement tray to the OR so that any delay in beginning or continuing the associated surgery can be minimized. Additionally or alternatively, the system may alter the schedule for when the surgery itself is set to begin. Still other actions related to OR scheduling and other matters may also be taken by the system based on the detection of the unsterile items and/or wet load.

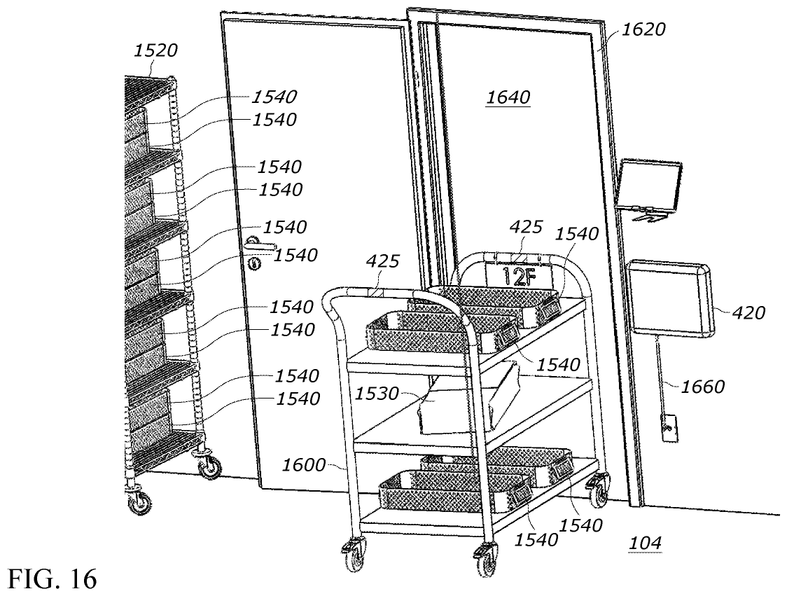

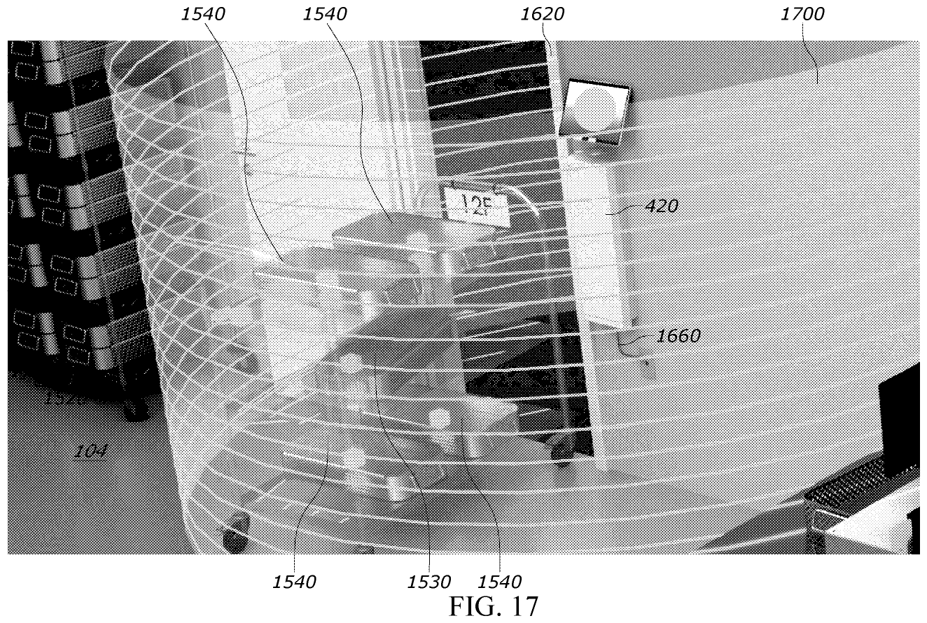

Now in reference to FIG. 17, this figure again shows the cart 1600 leaving the SPD 104, but with a magnetic field 1700 illustrated to demonstrate the sensing range of the sidekick device 420 to sense signals from the autoclave tags 500 on the cart 1600. It may thus be further appreciated from this figure that once inside the field 1700 around the doorway 1620, the sidekick device 420 may receive wireless signals from respective autoclave tags 500 on the respective trays 1530, 1540 as themselves located on the cart 1600.

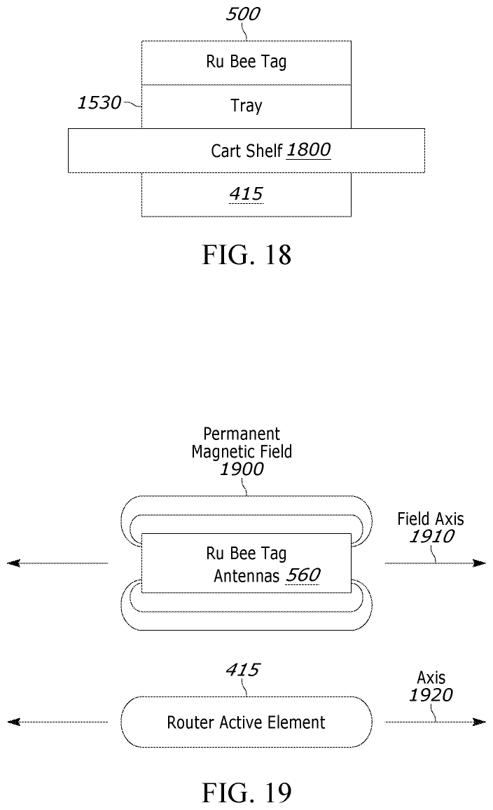

Again note that as mentioned above, to facilitate optimization of the sensing range of the magnetic fields used for the tags 500 and readers 405, 420, the respective antennas of the respective autoclave tags and readers may be aligned with each other. Refer to FIG. 18 for further understanding. This figure shows a side elevational view of a blue-wrapped sterile surgical tool tray 1530 with a tag 500 attached, with the tray 1530 sitting on a shelf 1800 of the rack 1520. FIG. 19 also shows the tag 500 and antenna 415 in isolation for further illustration.

As may be appreciated from these two figures, the field axis 1910 for the permanent magnetic field 1900 of the antenna 560 of the autoclave tag 500 is configured on the tray 1530 such that when the tray 1530 is positioned upright on the shelf 1800, the field axis 1910 is parallel to the axis 1920 of the antenna 415, at least in the X-Y plane even if the two antennas are positioned at different depths relative to each other. This allows the antenna 560 to bathe the antenna 415 in the magnetic field 1900. And if desired, to further aid in alignment, the shelf 1800 may be configured with physical contours that receive the tray 1530 in a particular orientation to maintain the antenna 560 at a same depth as the antenna 415 in the Z dimension while on the shelf 1800 for even greater alignment.

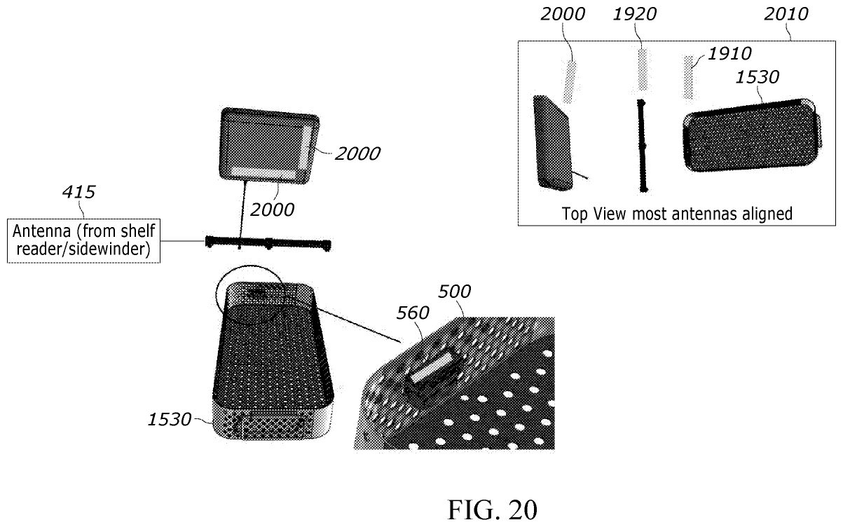

Similarly, the antenna(s) of the sidekick device 420 (another autoclave tag reader) as mounted on a wall near a doorway per the discussion above may also be mounted on the wall such that at least one of its internal antennas is aligned with the antenna 560 of a respective tag 500 on a respective tray as the tray is transported upright on a case cart. This is demonstrated in the schematic of FIG. 20, which schematically shows not just how the antennas 415 and 560 might be aligned with each other but also how at least one antenna 2000 internal to the sidekick device 420 may also be aligned with the antenna 560 of the autoclave tag 500. Also note that while the device 420 is shown as having two different antennas 2000 positioned with their long axes orthogonal to each other in the X-Y plane to sense autoclave tags 500 even when the tags 500 are in different orientations, even more antennas may be included in the device 420 in certain implementations.

Thus, as shown in FIG. 20, with two of the antennas 2000 oriented orthogonal to each other in the X-Y plane as the device 420 might be mounted on a wall, even if the associated tray itself is oriented at a different angle than upright as it passes by, one of the antennas 2000 will read the tag 500. And in many situations, the tray 1530 being placed upright on a case cart as it passes by the device 420 allows at least one of the antennas 2000 to align in the X-Y plane with the antenna 560 on the tag 500 as it passes by for optimized signal transmission. Further note that the inset 2010 of FIG. 20 shows a top view of the respective antennas with respective antenna long axes 1910, 1920, and 2020 aligned for further illustration.

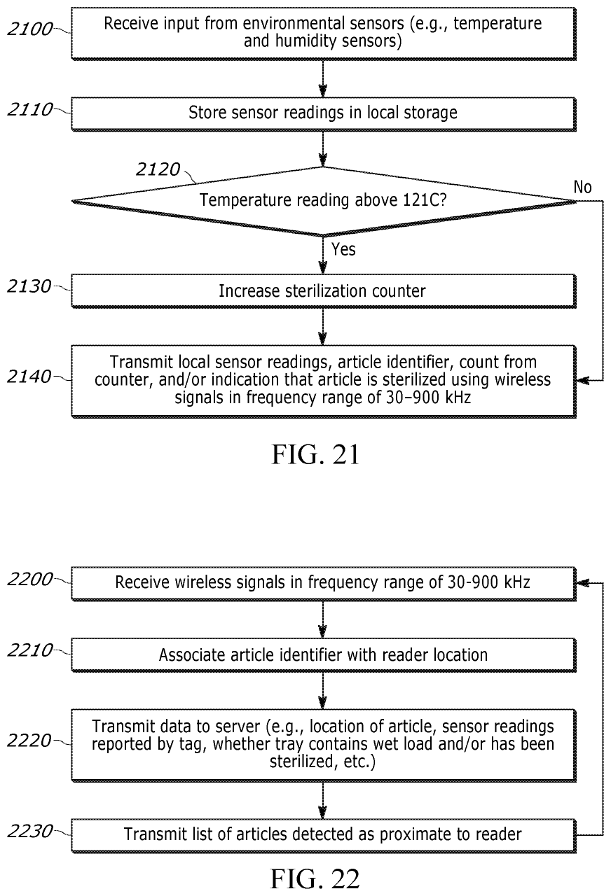

Referring now to FIG. 21, this figure shows example logic that may be executed by an IC in a tag 500 consistent with present principles, with it being reiterated that the IC may establish a processor system such as a dedicated microprocessor or another type of processor in non-limiting embodiments. Note that while the logic of FIG. 21 is shown in flow chart format, other suitable logic may also be used (e.g., state machine).

Beginning at block 2100, the IC may receive input from one or more environmental sensors on the tag 500, such as a temperature sensor and/or humidity sensor, that indicates readings from the sensors consistent with the disclosure above. The logic may then proceed to block 2110 where the IC may store the sensor readings in local storage of the tag 500. Next, the logic may move to block 2120 where the IC may determine whether the temperature reading reported by the temperature sensor has risen above a threshold temperature for a threshold amount of time (e.g., above 121° C. for thirty minutes), which may indicate that the article attached to the tag 500 has been sterilized as disclosed above.

A negative determination at diamond 2120 may cause the logic to proceed directly to block 2140 as will be described in a moment. But first, note that an affirmative determination at diamond 2120 may instead cause the logic to proceed to block 2130. Here the IC may increase its internal sterilization counter, as might also be stored in the tag's local storage, to indicate that an additional sterilization has occurred. The logic may then proceed to block 2140.

At block 2140 the IC may transmit the local sensor readings, article identifier for the associated article to which the tag is attached, current total sterilization count from the counter, and/or another indication that the article itself has been sterilized in the present instance. Again note that in non-limiting examples, the signals carrying that data may be sent in the frequency range of 30-900 kHz consistent with the IEEE 1902.1 standard, and even at 131 kHz in particular as that frequency is used in many instances for data transmission according to the IEEE 1902.1 standard.

Turning to FIG. 22, this figure shows example logic that may be executed by an IC in an autoclave tag reader such as device 405 or device 420 consistent with present principles, with it being reiterated that the IC may establish a processor system such as a dedicated microprocessor or other type of processor in non-limiting embodiments (e.g., central processing unit (CPU)). Note that while the logic of FIG. 22 is shown in flow chart format, other suitable logic may also be used (e.g., state machine).

Beginning at block 2200, the IC may receive wireless signals from one or more autoclave tags 500 via the frequency range of 30-900 kHz according to the IEEE 1902.1 standard (e.g., in the frequency of 131 kHz in particular). The logic may then proceed to block 2210 where the IC may associate an article identifier indicated in the signals with a current (known) location of the reader itself.

Next, the logic may move to block 2220 where the IC may transmit various types of data to a network node like the server 219 as discussed above. Thus, the data that is transmitted may include a location of the article associated with the received identifier, data indicating environmental sensor readings as reported by the tag 500, an indication of whether the associated article (e.g., surgical tool tray) contains a wet load as may be determined by the IC in the reader based on the reported humidity sensor readings from the tag 500, and/or whether the article has been sterilized as may be determined by the IC based on a report from the tag 500 and/or based on reported temperature readings and durations reported by the tag 500.

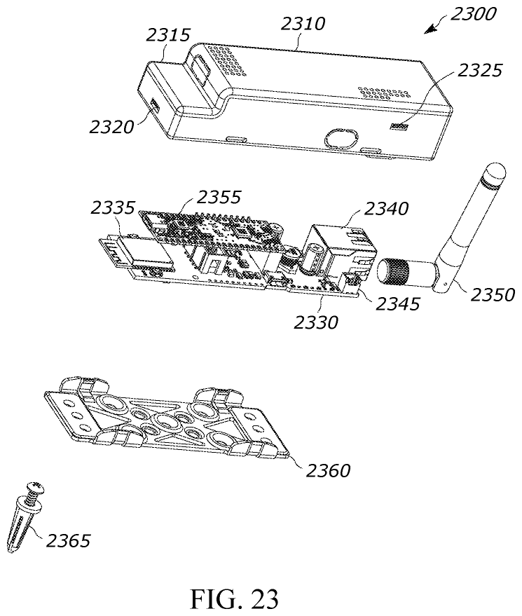

Continuing the detailed description in reference to FIG. 23, this figure shows an exploded view of an example BT reader 2300 that may be used consistent with present principles, where the BT reader 2300 may be the same or similar to the BT readers 215, 217, 325, and/or 400 described above. Note that a mesh of BT readers 2300 may be fixed to locations throughout a hospital or other healthcare facility to establish a mesh network of BT readers consistent with present principles.

As shown in FIG. 23, each reader 2300 may have a rigid housing 2310 that may be generally shaped as a rectangular prism with a step 2315 as shown. A universal serial bus (USB) port 2320 may be included on the front vertical face of the step 2315, such as a USB-C port, to provide an additional data connection to the reader 2300 when desired. The housing 2310 may also include one or more light emitting diodes (LEDs) 2325 that may illuminate while the reader 2300 is powered on to indicate as much.

As also shown in FIG. 23, the reader 2300 may include a PCB 2330 to which a variety of different components may be connected, such as a processor system 2335 (or other IC) as well other components accessible to the processor system 2335 like RAM, persistent storage, the LEDs 2325, the port 2320, a Wi-Fi network interface, an ethernet port 2340 for ethernet communication and POE power, and an antenna connector 2345 to connect to a BT/BLE antenna 2350. It is to therefore be understood that in at least some embodiments, the PCB 2330 may establish a POE Wi-Fi/BLE board that receives POE power via an ethernet cable plugged into the ethernet port 2340 (and/or includes other circuitry to otherwise receive power over a wired data communication line and to route the power to other components of the reader 2300). The PCB 2330 may also use the ethernet and/or other network connection to transmit, to a server of a healthcare facility, data indicating the location and identifier of various BT tags for which the reader 2300 receives BT signals.

Accordingly, a second BT board 2355 may be electrically and physically coupled to the PCB 2330 and accessible to the processor system 2355 to receive the BT signals themselves. The second BT board may also be embodied/disposed on its own PCB, and may be a BLE 6.0 board in particular and, as such, may include a separate BT transceiver. By including the second BT board 2355 on the reader 2300, a more robust hardware architecture is provided than would be realized using the Wi-Fi/BLE board 2330 alone as the BT board 2355 may be dedicated to transceiving BT signals while the PCB 2330 remains free for reliable, continuous high-speed Wi-Fi/HTTPs communication with other nodes on the network.

FIG. 23 also shows that a mounting bracket 2360 may be used to secure the reader 2300 to a surface via one or more set screws 2365. The mounting bracket 2360 may therefore be used to secure the device 2300 to a surface such as a ceiling or sidewall of a hospital for tracking of BT tags consistent with other potions of this disclosure.

In addition, note that while the reader 2300 may include two discrete boards 2330 and 2355 as described above, in other embodiments a single integrated board may be used in lieu of the boards 2330, 2355. The integrated board (and/or board 2330) may include a BT transceiver such as a BLE transceiver, a Wi-Fi transceiver, an ethernet transceiver for POE data and power supply, and even an ultrawideband (UWB) transceiver for UWB communication with tags and network nodes using UWB signals. This advantageously allows for the use of many different tags using a variety of communication protocols for high-fidelity location tracking of various articles within a hospital environment using a single integrated board, with UWB in particular affording particularly high-accuracy location tracking that might be especially helpful in indoor environments.

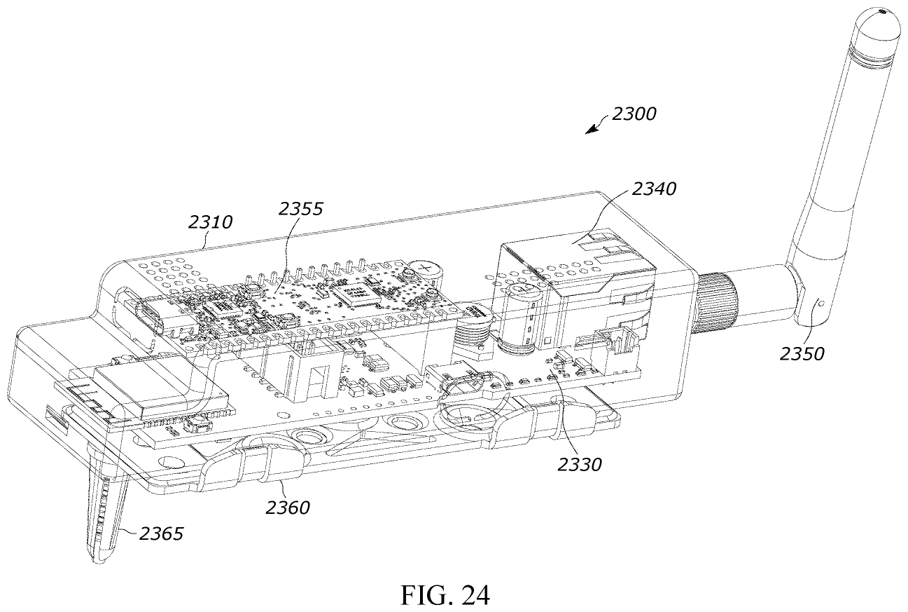

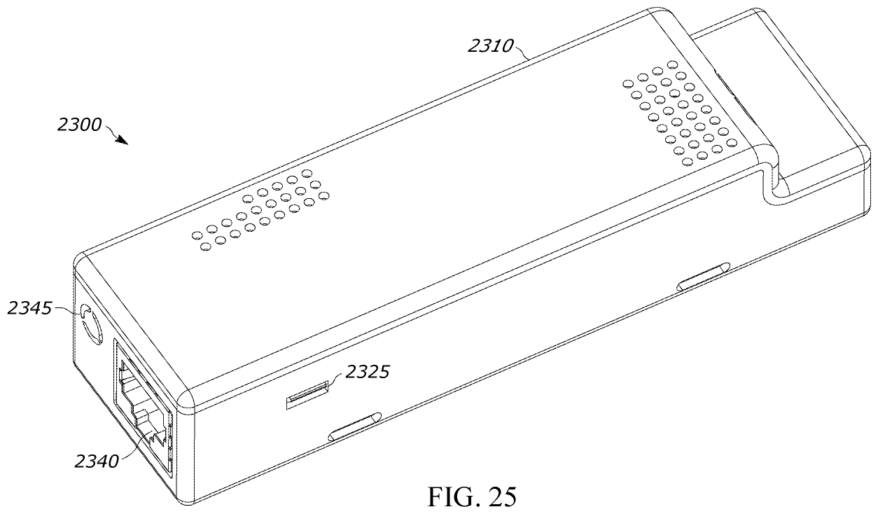

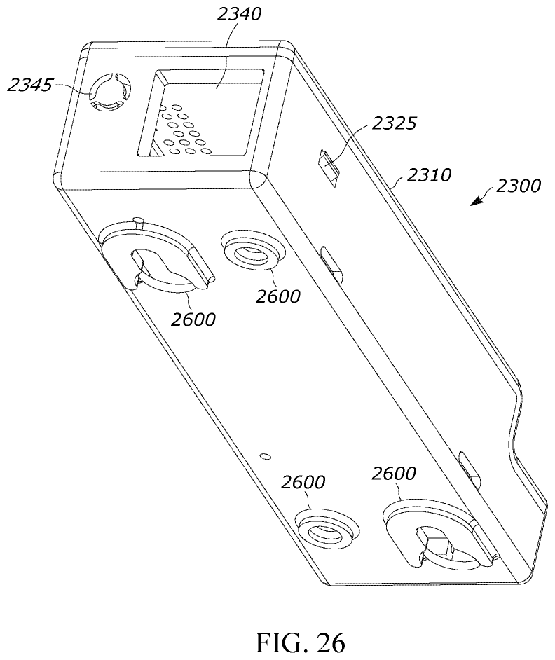

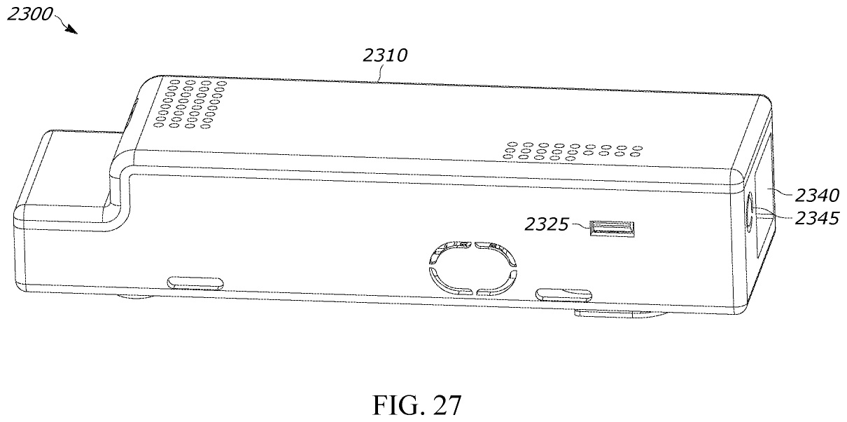

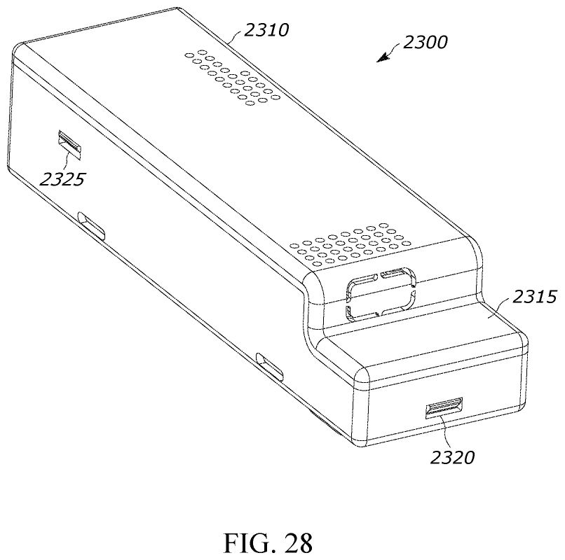

Turning now to FIG. 24, this figure shows a perspective transparent view of the reader 2300 as assembled and deployed in a hospital. FIG. 25 then shows an upper exterior perspective view of the reader 2300 from a first side, FIG. 26 shows a lower exterior perspective view, FIG. 27 shows an upper exterior perspective view of the reader 2300 from a second side, and FIG. 28 shows a front perspective view of the reader 2300, all sans antenna 2350 and bracket 2360 for simplicity. However, further note with respect to FIG. 26 that notches and nodules 2600 may be formed in the lower portion of the housing 2310 for snap or interference fit of the housing 2310 with the bracket 2360.

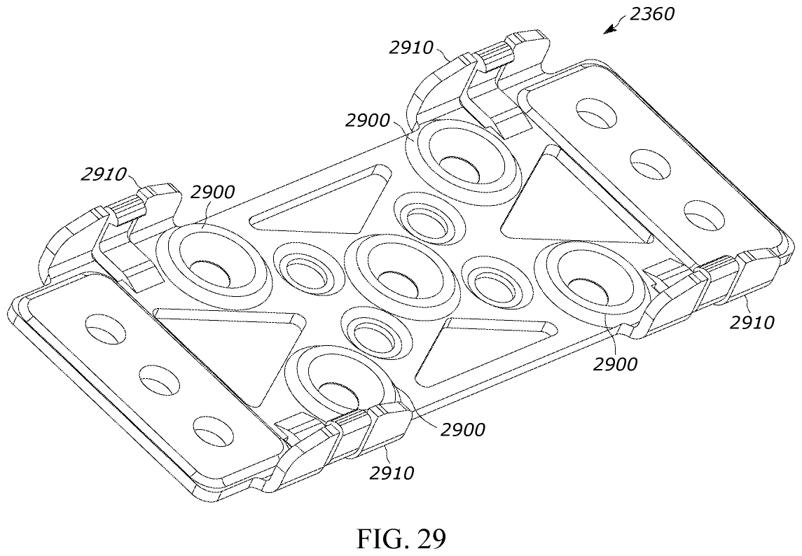

Now in reference to FIG. 29, this figure shows a perspective view of the bracket 2360 by itself. Note the moldings 2900 for snap or interference fit with the housing 2310 as described above. Also note the side arms 2910 that may wrap partially around the sidewalls of the housing 2310 in interference fit to further secure the housing 2310 to the bracket 2360.

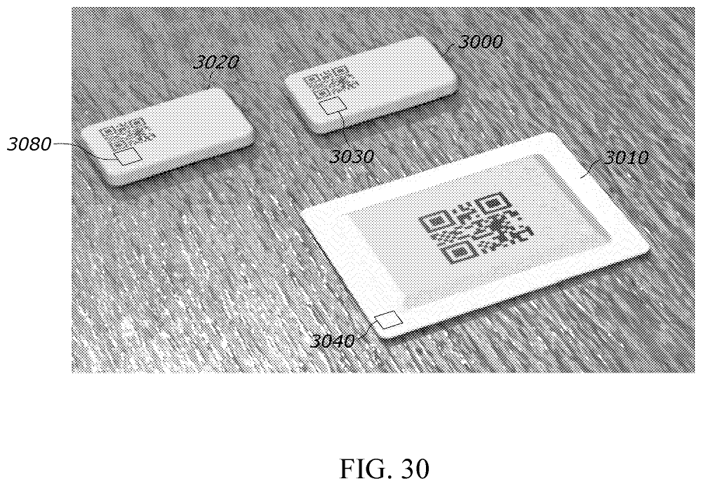

Moving on to FIG. 30, this figure shows perspective views of example BT tags that may be used consistent with present principles. The BT tags 3000 and 3020 may be similar to the BT tag 427 described above, while the BT sticker tag 3010 may be similar to the BT sticker tag 429 described above. The tags 3000, 3010, and 3020 are shown in close-up view, with it being understood that these tags may be sensed by the reader 2300 consistent with present principles. As such, may each tag 3000-3020 may have its own BT transceiver such as a BLE 6.0 transceiver in particular.

In addition, each tag 3000, 3010, and 3020 may have its own respective motion sensor 3030, 3040, and 3050. The IC in each tag may thus broadcast the respective identifier associated with its respective healthcare article responsive to one or more signals from its respective motion sensor indicating motion of the respective BT tag itself. It is also noted that each BT tag may also broadcast its respective identifier at a respective predefined time intervals so that each BT tag's location can still be monitored even if it stays stationary. In any case, the motion sensor on each BT tag may be an accelerometer in one non-limiting embodiment, though it might additionally or alternatively be a gyroscope, magnetometer, or another type of motion sensor. In one particular example, each BT tag may include an inertial measurement unit (IMU) including at least both of a respective accelerometer and respective gyroscope, and even a respective magnetometer.

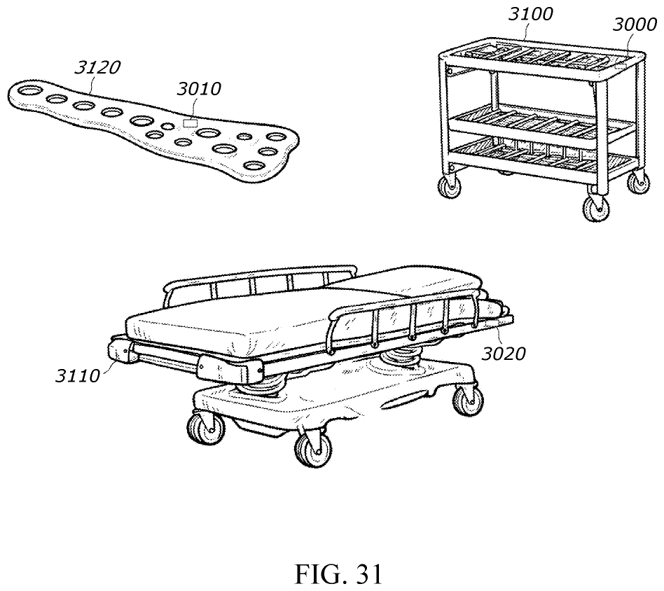

Next, FIG. 31 shows the BT tag 3000 affixed to a surgical case cart 3100, the BT tag 3020 affixed to a surgical bed 3110, and the BT tag 3010 affixed to a medical implant 3120. Note that the BT tags 3000, 3020 are affixed to the case cart 3100 and surgical bed 3110, respectively, as those two articles may be used repeatedly within a hospital and therefore the BT tags 3000 with relatively longer battery life may be affixed thereto, while the BT sticker tag 3010 is affixed to a single-use implant and therefore need not have as long a battery life (e.g., with the sticker tag 3010 being included on packaging or an otherwise removable pat of the implant 3120 prior to implantation in a patient).

Further note that the BT tag 3000 may store a first unique identifier identifying the surgical case cart 3100, with the BT tag 3000 configured to communicate with a plurality of BT readers spaced apart from each other in the healthcare facility as discussed herein (e.g., a plurality of BT readers like the reader 2300 that establish a mesh network configured to track the current location of each BT tag as the respective BT tag moves about the healthcare facility). In addition, the BT tag 3020 stores its own unique identifier identifying the surgical bed 3110, with the BT tag 3020 also configured to communicate with the plurality of BT readers. Further still, the BT tag 3010 stores yet another unique identifier identifying the medical implant 3120, with the BT tag 3010 also configured to communicate with the plurality of BT readers.

Before moving on, note with respect to unique identifiers that might be transmitted by BT tags, autoclave tags, or even other types of tags that the identifiers themselves may take numerous different forms yet still uniquely identify the respective item to which they are attached. Accordingly, as non-limiting examples, each identifier may be a 48-bit Bluetooth Device Address (for BT tags), a media access control (MAC) address, a universally unique identifier (UUID), or even a vendor-specific identifier.

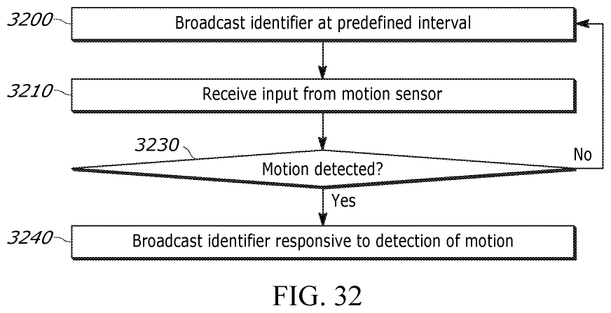

Continuing the detailed description in reference to FIG. 32, this figure shows example logic that may be executed by an IC in each BT tag 3000, 3010, and 3020 consistent with present principles, with it being reiterated that each IC may establish a processor system such as a dedicated microprocessor or other type of processor in non-limiting embodiments. Note that while the logic of FIG. 32 is shown in flow chart format, other suitable logic may also be used (e.g., state machine).

Beginning at block 3200, the IC of the BT tag may broadcast its unique identifier for a healthcare related article associated with the BT tag itself as already stored in local storage of the BT tag. Here the broadcast of the identifier may be using the BLE 6.0 protocol, for example, and may be a broadcast occurring at a predefined time interval regardless of whether the BT tag undergoes motion or remains stationary.

From block 3200 the logic may then move to block 3210. Here the IC in the BT tag may receive input from the BT tag's on-board motion sensor(s) to determine, at decision diamond 3230, whether motion of the BT tag itself has been detected based on the input. A negative determination may cause the logic to revert back to block 3200 for the IC to continue broadcasting the BT tag's unique identifier at the predefined time interval (e.g., every ten seconds).

However, an affirmative determination may instead cause the logic to proceed to block 3240 responsive to motion being detected. Here the IC may broadcast additional signals indicating the BT tag's identifier via additional BT signals (different from those transmitted at the predefined intervals). From block 3240 the logic may then revert back to block 3200 for the IC to continue broadcasting the BT tag's unique identifier at the predefined time interval, with it being further noted that the BT tag may also continuously broadcast its unique identifier as long as motion continues to be detected for high-fidelity location tracking as the tag moves about the hospital.

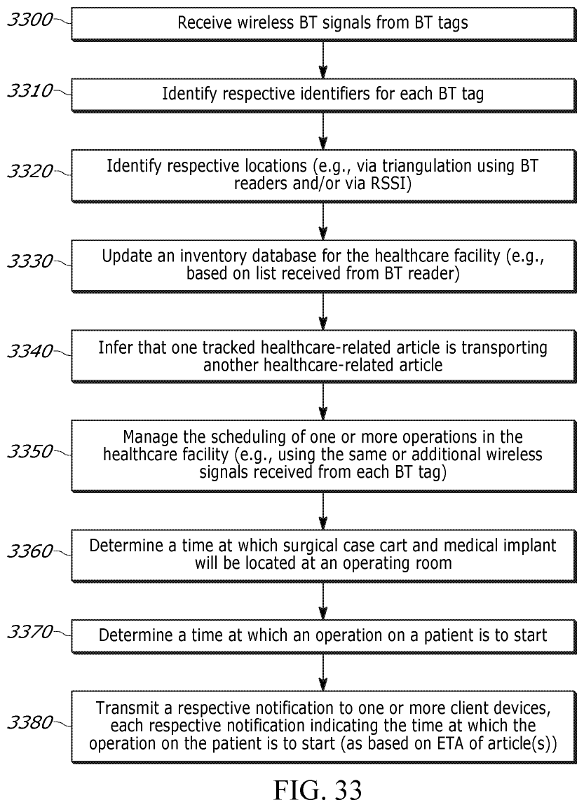

Now in reference to FIG. 33, this figure shows example logic that may be executed by an IC in at BT reader and/or by a server in communication with the BT reader consistent with present principles (e.g., HTTPS communication). Thus, various steps in the logic may be performed in whole in or in part by either one of the BT reader and/or the server in any appropriate combination, though one or the other of these devices might be described below as performing a particular function as an example. Note again that while the logic of FIG. 33 is shown in flow chart format, other suitable logic may also be used.

Beginning at block 3300, one or more of the mesh network of BT readers (e.g., one of the readers 2300) may receive wireless BT signals from one or more BT tags (e.g., BLE signals according to the BT 6.0 protocol). The logic may then proceed to block 3310 where the reader may identify, from the received signals, a respective identifier transmitted by each BT tag for which signals have been received.

The logic may then proceed to block 3320. Here the BT reader (or server with which the BT reader is communicating) may identify respective locations of the respective BT tags for which signals have been received at block 3300. For example, the server may execute one or more triangulation algorithms using signals for the same BT tag but as received by different BT readers at different locations within the healthcare facility to triangulate the location of the respective BT tag. Additionally or alternatively, the server and/or BT reader may execute one or more received signal strength indicator (RSSI) algorithms to identify the location of the respective tag. Thus, in one particular instance, RSSI algorithms may be executed to identify the location of the respective BT tag based on signals received from the BT tag at different BT readers so that not only distance but also direction can be inferred from the signals using RSSI. In addition, in some examples one or more channel sounding algorithms, phased-based ranging algorithms, and/or round trip timing (RTT) algorithms may be used to identify the location of the BT tag.

After block 3320 the logic may proceed to block 3330 where the server may, based on signals received from respective BT tags, update an inventory database for the healthcare facility. In one particular instance, the server may receive a list of healthcare articles for which respective signals have been received and identified by a respective BT reader within a threshold amount of time of a current time, with the list indicating the healthcare-related article associated with each received identifier. The database may then be updated based on each list as received from a respective BT reader.

In various examples, updating the database may include indicating in the database that a particular healthcare-related article associated with a given BT tag for which signals have been received has been taken from a storage location and therefore will be used. Additionally or alternatively, updating the database may include reducing an inventory count for the associated article type in the database based on one of the associated articles being removed from the storage location as inferred from movement of its respective BT tag.

The logic may then continue to block 3340. Here the server may use the location of a sensed BT tag and a reported location of another tag (e.g., autoclave tag 500) to infer that a first healthcare-related article to which the BT tag is attached (e.g., a case cart with an attached hybrid tag 435 with BT transceiver) is transporting a second healthcare-related article to which the other tag is attached (e.g., a surgical tool tray). This may be done as set forth above, e.g., by determining that the location of the BT tag and the location of the other tag are within a threshold distance of each other at a same or similar time of day.

Based on the inference, the logic may then proceed to block 3350. Here, based on the inference, the server may manage the scheduling of one or more operations in the healthcare facility based on the signals received at block 3300 and/or additional BT signals received as the respective BT tags continue to move throughout the facility. For instance, the server may schedule a surgery in a hospital's electronic calendar based on a projected estimated time of arrival (ETA) at a particular operating room for the articles associated with the case cart and surgical tool tray in the paragraph above. The server might also take one or more actions to notify one or more healthcare personnel of the ETA of the surgical tool tray and/or an associated surgery start time that is based on the ETA.

In terms of the ETA, while the ETA may be determined using rules-based algorithms, such as those that might account for average speed of the tags as sensed by the readers as the tags move about the healthcare facility over a known distance and toward a designated operating room, AI models may also be used to determine as much for even greater precision. For instance, the system may track different tag travels over time to assemble a dataset that includes total travel times for different article types along different known travel paths within the healthcare facility that the articles too, with the dataset then being used to train a pattern recognition model such as a recurrent neural network or feed forward neural network to make additional ETA inferences in the future (for the same article type) based on the past travel times from the dataset. In some instances, the model may even be configured to recognize patterns for certain geolocations within the healthcare facility where an article's movement typically temporarily stops, such as at an elevator bank or badge check point, to factor those locations and the associated time delays into future ETA predictions for future tag travel known to go past the same geolocation.

Accordingly, at block 3360 the server may use location tracking and the predicted ETA to determine a particular time at which a surgical case cart, tool tray, and/or medical implant will arrive at an operating room. Based on that, at block 3370 the system may also determine another time at which an associated operation on a patient is to start in that operating room. In some examples, those two times may be the same (ETA and surgery start time). However, to provide a buffer in case there is an unexpected delay in the arrival of those items, the operation start time may be no more than a threshold amount of time after the items'arrival time at the operating room. This might be particularly useful where the predicted arrival time is an earliest time at which the surgical case cart and the medical implant will be located at the operating room as determined based on current movement the BT tags attached to those two items (e.g., and execution of a pattern recognition model as set forth above).

From block 3370 the logic may then proceed to block 3380. Here the server may, based on the determination of the time at which the operation is to start, transmit a respective notification to one or more client devices. Each respective notification may indicate the ETA and/or the time at which the operation on the patient is to start, where each client device may be associated with a different person assigned to participate in the operation itself (e.g., one or more surgeons, one or more nurses, one or more physician assistants, etc.). In some examples, like if a particular operating room had not yet been assigned for the operation, the notification sent to each client device may also indicate an identifier for the particular operating room to which the personnel are to go (e.g., OR #5). The notifications may be presented audibly and/or visually at each client device using a speaker and display, respectively.

In addition to notifying the aforementioned client devices, the server may also send notifications and/or control commands to one or more perioperative robots that are also scheduled to assist in the surgery (e.g., humanoid robots). Thus, the robots may be commanded to move and arrive at the designated operating room, sterilized, at or before the designated operation start time.

Also at block 3380, the server may communicate with the client device(s) to present other graphical user interfaces (GUIs) on each device's display. For instance, a GUI may be presented that indicates the respective locations of the respective tags that are being tracked, and therefore the respective locations of the associated articles themselves. The GUI might therefore present, for example, a blueprint or hospital map that includes markings for the respective current locations of each tag and/or associated article.

Still in reference to FIG. 33, it is to be understood that still other actions may be taken by the server based on the tracking of various BT tags and/or autoclave tags (more generally, wireless communication tags) throughout the healthcare facility. For instance, based on the current inventory level as tracked by the server according to the description above, the server or robot can order additional articles for upcoming surgeries for which required items are currently unavailable as determined from the current inventory count.

The server may also track the locations of trays, tools, carts, and other items to identify any such articles that will not be available for other surgeries even further in the future. In addition, depending on when additional supplies are estimated to be available, any such future surgeries may even be rescheduled for a later time. Thus, additional articles may be ordered in advance of surgeries when inventory is low, and surgeries may even be rescheduled far in advance when the system knows certain required articles will not be available (potentially freeing up the OR for a different type of surgery for which required articles are currently in-stock to optimize OR usage). These things may also be determined based on execution of a pattern recognition AI model (e.g., RNN or feed forward neural network) that can be executed to do a historical analysis of the uses of trays, robot, case carts, tools, how long a patient was in the room for different respective surgeons that operate at difference paces, etc. in the past to then determine future scheduling and whether additional articles should be ordered or otherwise provisioned based on the current inventory counts and scheduled surgeries.

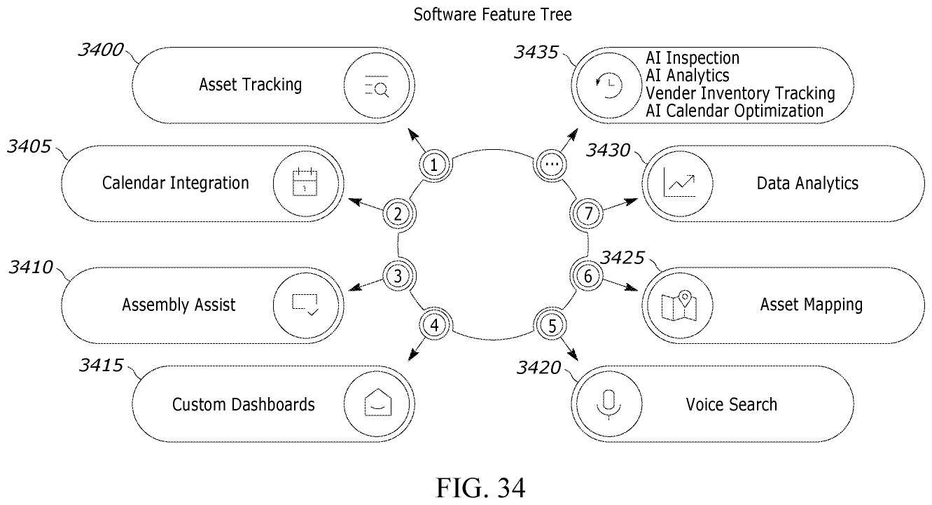

Continuing the detailed description in reference to FIG. 34, this figure shows an example software feature tree consistent with present principles. As shown, asset tracking software 3400 may be implemented as discussed herein and may interface with a calendar integration module 3405. If desired, assembly assist software 3410 may also be used to inform hospital personnel and/or a perioperative robot of what specific tools should go into a tray that is to be transported to an OR, and even what blue-wrapped trays themselves should be transported. Custom dashboards 3415 including the GUIs referenced herein may also be implemented, along with voice searching software 3420 which may be used for hospital personnel to provide voice input (via a client device) asking for article status and location, which may then be reported back to the person via audible output based on the location tracking techniques disclosed herein.

In addition, the software feature tree of FIG. 34 also demonstrates that asset mapping software 3425 may be used to track articles as they move about the hospital as described above, with the software 3425 also able to output a map showing the real-time location of various assets consistent with this disclosure.

Data analytics software 3430 may also be implemented to analyze various data reported by various sensors of the tracking system. Thus, the software 3430 may store and analyze records including reported identification data for each article, such as the type of tool, quantity, sterilization cycle information, location data within the hospital infrastructure, and usage history. The software 3430 may also retrieve this data responsive to personnel or robot request, employing querying functionalities for real-time access to inventory statuses and history, ensuring up-to-date information is available to hospital staff and hospital robots alike.

FIG. 34 also shows that the software feature tree may include AI models 3435, including those discussed above. As other examples, AI inspection models and AI analytics models may be used to inspect and analyze records for articles tracked consistent with this disclosure. Vendor inventory tracking AI models may also be executed to track inventory as categorized by specific vendor to which certain inventory within the hospital belongs.

In addition, an AI calendar optimization model may be executed to optimize OR booking times, scheduling surgeries as close together as possible using the high-fidelity tracking system discussed herein to ensure that hospital resources are being used as effectively as possible. Thus, various types of pattern recognition models may be used such as RNNs, feed-forward neural networks, reinforcement models, and/or transformer models.

In addition, transformer models such as generative pre-trained transformers may also be used to provide outputs on GUIs to hospital personnel (and electronic notifications to hospital robots) that are related to asset locating, asset history/logging, cart checkout procedures, and inventory management. Such AI models (and others) may even be used to present GUIs with suggestions on various trays for hospital personnel to load onto a case cart for surgery (and even a particular location on the case cart on which to put the tray), as well as directions to precisely locate any such trays within the SPD for loading onto the cart (e.g., as located on a storage rack at a particular side of a particular shelf). Indications may also be presented for returning trays to a particular storage rack after sterilization. Storage rack locations might be indicated by storage rack identifier and shelf identifier using text, for example. Also, tray and tool status indicators may be presented for associated as well—e.g., “dirty”, “clean/sterile”, or “in process”. Further note that these GUIs may be presented on a non-transparent display of a mobile device and/or on the transparent display of an AR headset, where the AR headset shows the location according to the current bearing of the user, whether in text form or using a non-text graphical indicator such as a bulls-eye.

These AI models may also be used to flag (notify) certain things for hospital personnel and perioperative robots alike, including inventory deficiencies, priority rushes, missing tray indicators, missing tool indicators, and surgical holds placed by robots and others. Total inventory for each item may also be presented as another indicator, such as responsive to an audible request to view inventory for the hospital. Moreover, respective locations may be presented for itemized inventory items presented on a client device display.

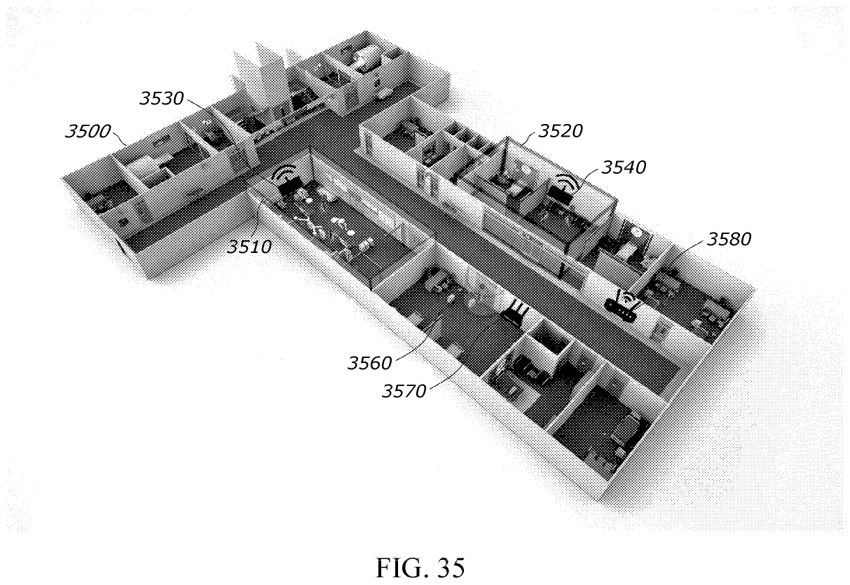

Now in reference to FIG. 35, this figure shows an example hospital layout of a hospital 3500 to further illustrate present principles. As shown, various room zones 3510, 3520 for individual hospital rooms may be monitored via respective BT readers 3530, 3540. In addition, a three-dimensional zone 3550 in real space around a doorway of another room may be monitored by an autoclave tag reader 3570 similar to the reader 420. Also note that a Wi-Fi router 3580 may also be located within the hospital 3500 to provide additional network connectivity to readers such as the readers 3530, 3540, and 3570 (in addition to other devices connected to the same network). And note that while readers are shown in only certain rooms as an example, both BT readers and autoclave tag readers may be placed in each room of the hospital 3500 if desired.

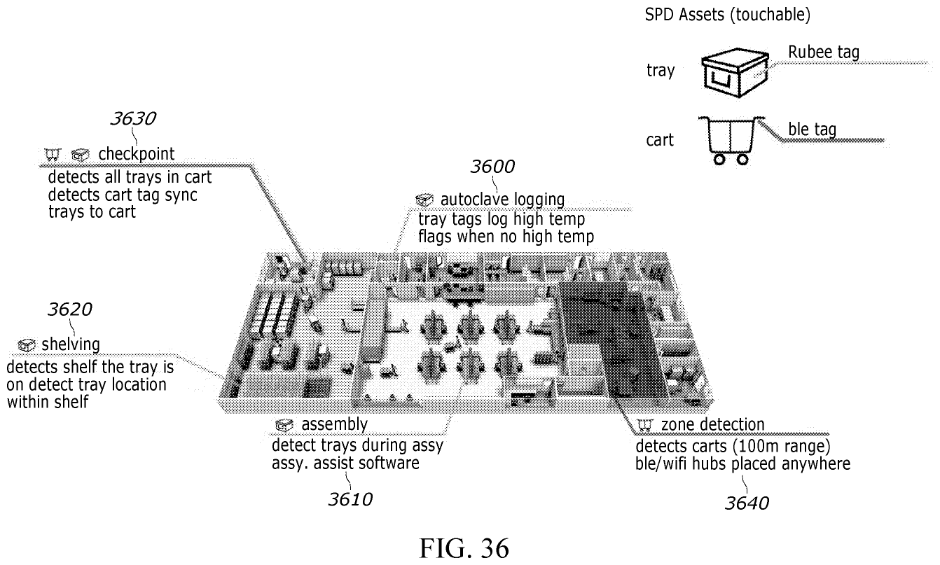

Turning to FIG. 36, an example SPD layout is shown along with process flow indicators to further illustrate present principles. At a first step 3600, respective autoclave tags may log sensed high temperatures during autoclave sterilization as set forth above, and may also store flags when no high temperature is sensed as expected during the autoclave process. As the respective tag exits the autoclave, at step 3610 autoclave tag readers may detect trays during their assembly, where various sterilized surgical tools are placed inside the trays using assembly assist software (indicating to robots and/or personnel what tools should go into what trays as already assigned to future surgeries).

Subsequently, at step 3620 the sterilized trays (with tools inside and already blue-wrapped to maintain sterilization) are placed on shelves of storage racks consistent with the disclosure above. Here autoclave tag readers on the shelves may detect respective shelves on which each tray has been placed and even detect the tray's location on the shelf itself. Then, at step 3630, once the trays have been taken back off the shelves and placed on case carts for future surgeries, a reader checkpoint detects all trays in a given case cart on which the trays have been placed and detects the cart tag itself (e.g., BT tag) to synchronize the location of the trays to the cart itself. Further note that during all of this, zone detection 3640 may be performed to detect the location of the carts themselves (e.g., with a 100 meter range) using BLE and/or Wi-Fi hubs placed in the SPD as the carts move about the SPD.

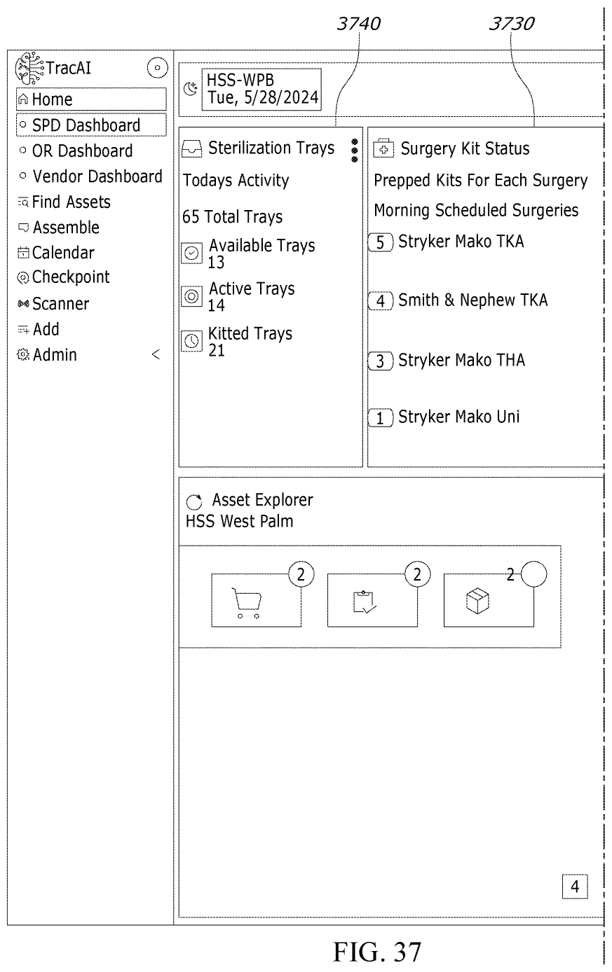

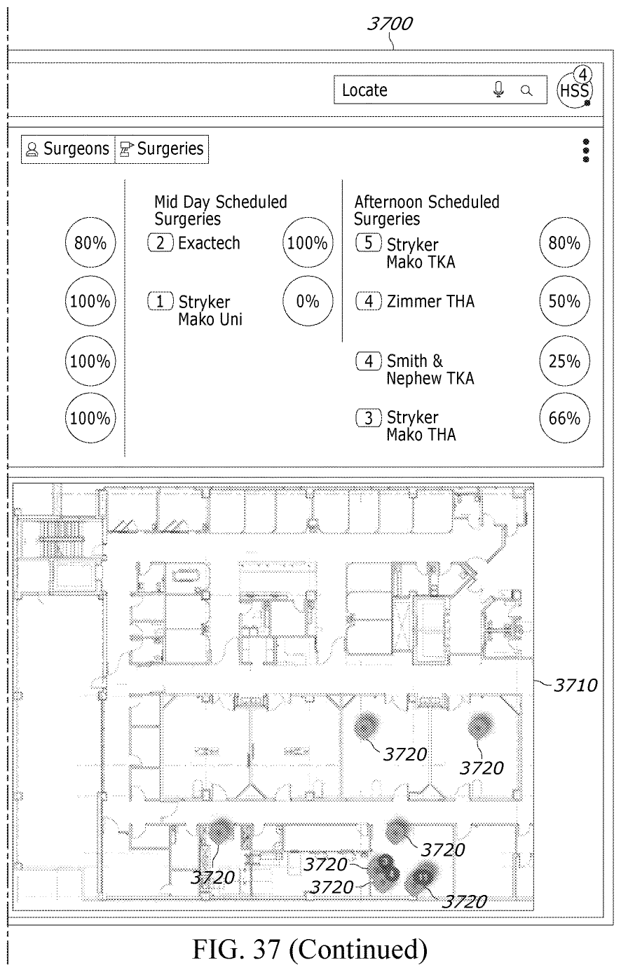

Continuing the detailed description in reference to FIG. 37, an example GUI 3700 is shown that may be presented at a client device and/or robot display for hospital personnel to view real-time current locations of various healthcare-related articles as they move about a hospital. Accordingly, the GUI 3700 may include a hospital blueprint 3710. In addition, arrows or other graphical indicators 3720 may be placed at various points on the blueprint 3710 at which associated articles that have been sensed are currently located in the real-world. Note that some of the indicators 3720 may have a number or other identifier assigned to them so that the particular article and/or article type can be determined using the legend 3730.

An additional data dashboard 3740 may also be included on the GUI 3700. The dashboard 3740 may indicate inventory counts for various types of articles, such as available trays and even trays actively in use at the current time. Trays that have already been sterilized and kitted with sterile tools may also be indicated as a separate entry if desired.

In some examples, additional input elements may be included on the GUI 3700 to enable hospital personnel to perform inventory searches, view inventory records, generate inventory reports, and receive inventory alerts based on criteria set forth by the personnel. These features can be used for tracking the flow of assets and identifying potential bottlenecks or inefficiencies in the asset management process.

It may thus be appreciated that the GUI 3700 presents location and status data in a structured and user-friendly manner, allowing hospital staff to quickly ascertain the availability, location, and sterilization status of surgical tools. This facilitates effective planning for surgical procedures, reduces incidences of tool shortages, and enhances the hospital's responsiveness to emergent needs.

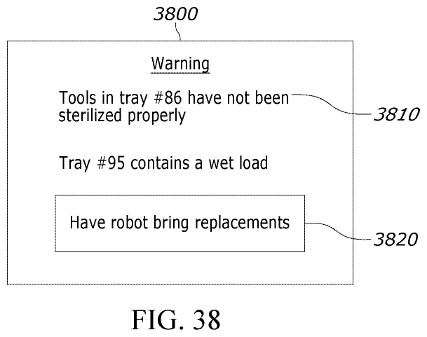

Turning to FIG. 38, another example GUI 3800 is shown that may be presented on the display of a client device and/or robot. The GUI 3800 may be presented to warn hospital personnel that certain trays have not been sterilized as intended and/or that those trays contain wet loads. Accordingly, indications 3810 may not only indicate as much but also identify the particular tray(s) to which the warning message(s) pertain (by unique tray number per this example). In some examples, the GUI 3800 may also include a button 3810 that is selectable via touch input to command a hospital robot to go fetch replacement trays and/or tools for the ones that have been determined by the system as unsterile and/or wet.

Moving on from FIG. 38, it is to be understood that just as certain articles are tracked within a healthcare facility as mentioned above, those articles may also be tracked at an off-site warehouse where the tools and trays might also be sterilized, stocked, etc. Thus, here articles may be tracked from warehouse to vehicle to hospital and back again, providing high-fidelity location tracking throughout the cycle of the articles as they are used over and over again.

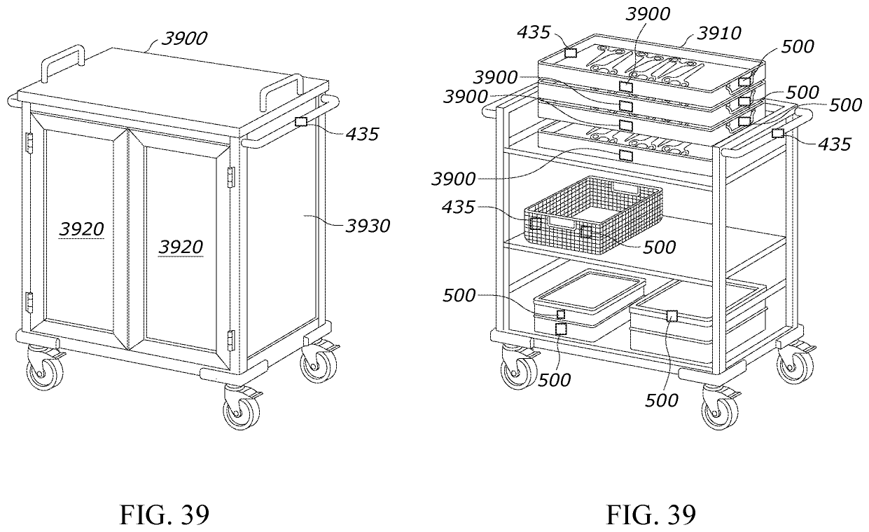

Now in reference to FIG. 39, this figure shows two example case carts 3900, 3910 that may be implemented consistent with present principles to transport surgical tool trays and other items between locations within the hospital. Note that the cart 3900 is an enclosed case cart where metal doors and walls 3920, 3930 enclose tool trays (with autoclave tags 500) located inside, while the cart 3910 has its side areas open as also shown.

It may be appreciated from this figure that not only may autoclave tags 500 use the IEEE 1920.1 standard to successfully transmit signals to autoclave tag readers external to the carts when the carts are open (like the cart 3910), but may also successfully transmit signals to autoclave tag readers external to the cart even when the cart is enclosed on all sides by metal that might block the transmission of other types of signals (like for the cart 3900). The autoclave tags may also allow for reading through the fenestrations in the trays themselves, regardless of which cart the trays are on.

Further note that one or more hybrid tags 435 may be included not only on the carts 3900, 3910 but also on certain trays, such as those on the open cart 3910, so that the trays on the cart 3910 can be tracked using BT and 1902.1 communications in tandem (e.g., in embodiments where the tags 435 also have potting and other features to protect the BT components therein from the heat of the autoclave). In addition, note that in some examples the identifiers from the tags 435 for the carts 3900, 3910 may be used for the system to identify and indicate to robots and humans which particular trays should be loaded onto which cart based on the cart's identifier and that cart being assigned to transport those particular trays.

What's more, it is to be understood that one or more trays may include unique barcodes and/or quick response (QR) codes 3900 as also shown in FIG. 39. These codes may be scanned by people and/or robots with barcode/QR code readers for additional confirmation that an associated tray is in fact a tray intended to be transported. As such, the barcode or QR code 3900 may indicate the same identifier as the one indicated by the same tray's respective autoclave tag 500.

In addition, for embodiments where a perioperative humanoid robot is the entity taking trays off storage shelves and loading them onto the case cart to take the trays to an OR for surgery, the robot may execute computer vision to view the barcodes or QR codes 3900 using an on-board camera and identify the trays by the respective identifier indicated via the code(s) 3900. And since the robot may include its own autoclave tag reader as well, the robot can determine that it has the correct tray through both sensing modalities (autoclave tag signal reception and computer vision) for added redundancy to ensure the right trays are brought to the OR so that the surgery can transpire as scheduled.

Thus, per one example, the robot may identify, via autoclave tag, the particular shelf of a storage rack on which a particular tray is stored that is to be taken by the robot. The robot's vision model may then be used to read the barcode or QR codes 3900 of each tray on that shelf to identify the particular tray on that shelf that should then be placed on one of the case carts 3900, 3910, improving overall specificity and ensuring an incorrect tray is not taken.

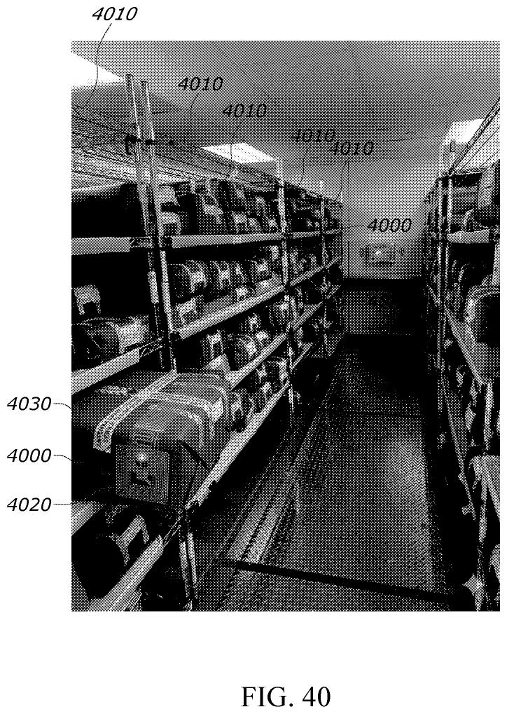

Now in reference to FIG. 40, it is to be understood that autoclave tags 500, and/or at least a respective antenna 560 with copper winding 565, may be integrated into blue wrapping that is used to enclose tools and trays that have already been sterilized. FIG. 40 thus shows various blue-wrapped trays 4000 that are located on various storage racks 4010 within an SPD. One of the trays 4000 is shown in the foreground of this figure with an autoclave tag antenna 4020 integrated into its tray blue wrapping 4030. Further note that additional autoclave tag antennas may also be incorporated into tape that is used to seal the blue wrap 4030 as another sensing means.

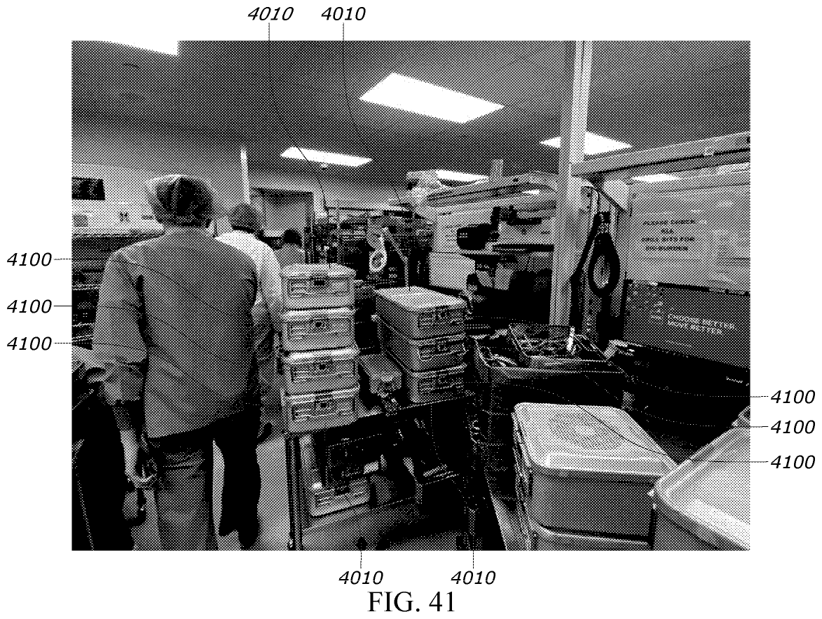

Additionally, as shown in FIG. 41, autoclave tags and/or at least their antennas may be incorporated into reuseable orange clips 4100 that are used to confirm the lid of various tool trays 4010 are sealed.

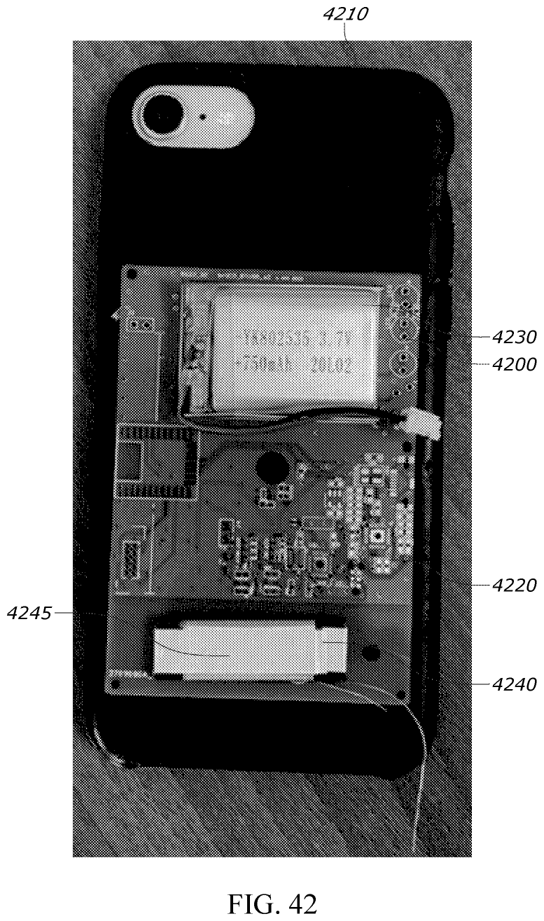

Now describing other aspects that may be implemented consistent with present principles, a mobile autoclave tag reader may also be embodied on a client device such as a smartphone consistent with present principles so that this tag reader may also read autoclave tags via their respective antenna emissions, whether those antennas are on the trays, blue wrap, tape, orange clip, etc. FIG. 42 therefore shows an example autoclave tag reader 4200 as wired to the circuitry of a client device 4210 (e.g., smartphone).

As shown in FIG. 42, the reader 4200 may include an IC 4220 that may be similar to the IC 1420, and a battery 4230 to power the reader 4200 (including IC 4220). The reader 4200 may also include an autoclave tag reader antenna 4240 with a copper wire winding 4245 that may be used to receive the wireless signals from various autoclave tags and provide those signals to the IC 4220.

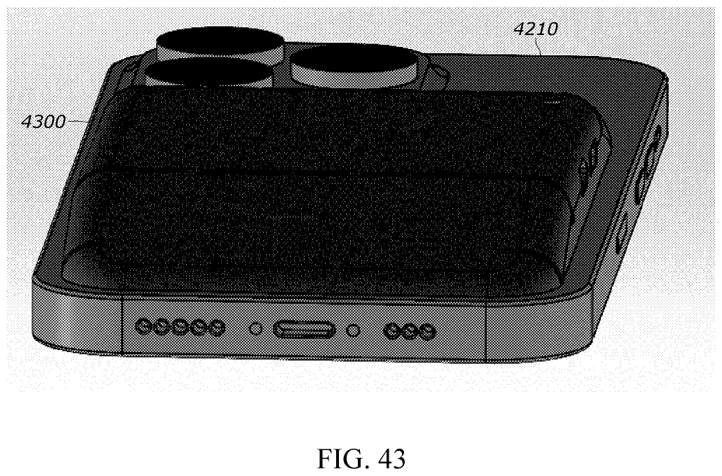

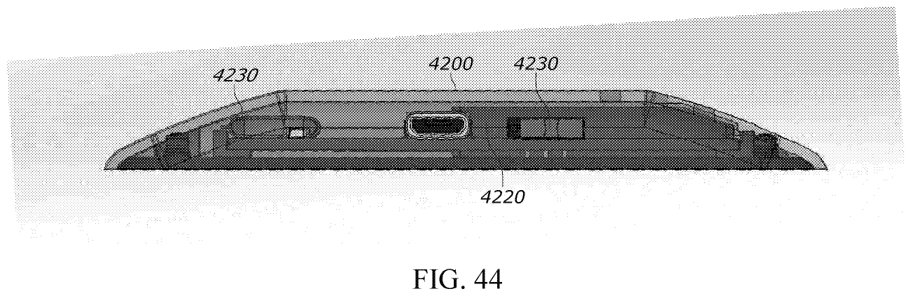

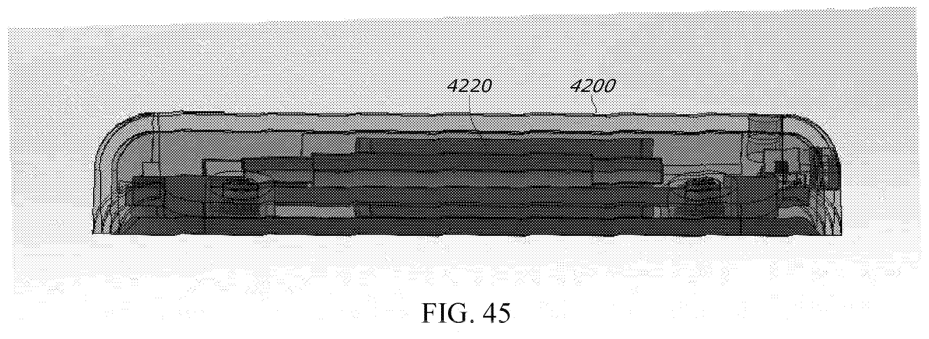

FIG. 43 then shows the reader 4220 in perspective view, protected by a rigid reader housing 4300 and attached to the client device 4210. FIG. 44 also shows the reader 4220 in bottom cutaway view while FIG. 45 shows the reader 4220 in top cutaway view.

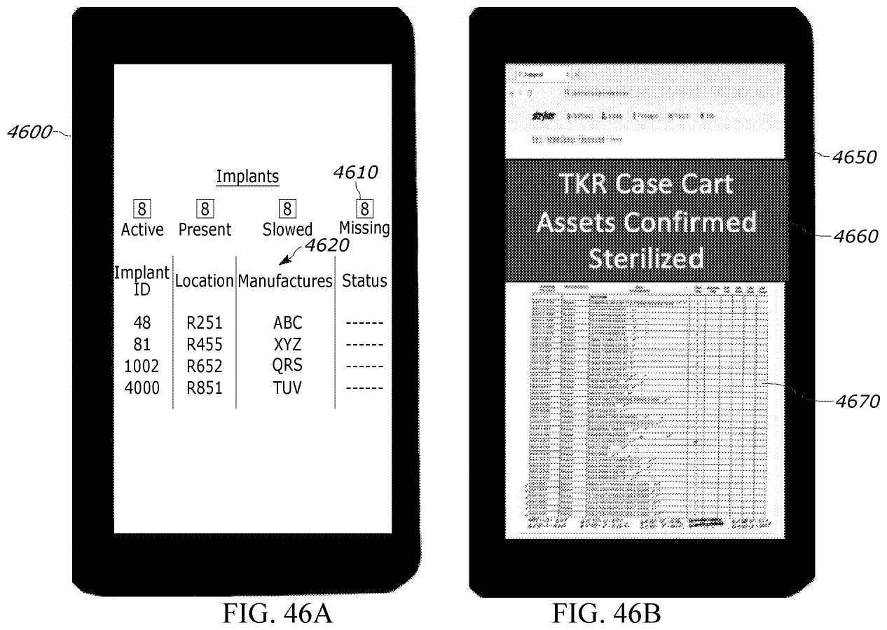

Next, FIGS. 46A and 46B show GUIs that may be presented on the display of the client device with autoclave tag reader 4200. Beginning with FIG. 46A, responsive to the client device's reader having scanned autoclave tags within range and recognized their implants and/or trays, the GUI 4600 may be presented. As shown, the GUI 4600 may include indicators 4610 indicating respective particular amounts of autoclave tags that are respectively active, present, misplaced, and missing. The GUI 4600 may also include a table 4620 with columns listing, for each autoclave tag that was recognized, the associated implant's unique identifier, a location of that implant within the SPD by both rack number “R” and shelf number “S”, the manufacturer of each implant, and additional statistics and data about each implant.

Turning to FIG. 46B, now suppose a robot or person has used an autoclave tag reader to identify various surgical tool trays to load onto a case cart, and has then loaded the trays needed for an upcoming surgery onto the cart itself. The robot or person might then use its autoclave tag reader to read all the autoclave tags on the cart again for added confirmation. For humans in particular, the human might provide voice input to read the tags. Then in response to reading the tags again, the GUI 4650 of FIG. 46B may be presented on the display of the client device 4210 in response.

As shown, the GUI 4650 may include an indicator 4660 that confirms based on the data from the autoclave tags (e.g., humidity and temperature data) that all the articles in each tray monitored by a respective autoclave tag have been confirmed as sterilized. A list 4670 might also be presented that lists all the articles identified via autoclave tag identification as being located on the cart for additional visual confirmation by the robot or person.

Now in reference to FIG. 47, as mentioned above, there may be instances where a perioperative humanoid robot is responsible for assembling a case cart with certain sterilized trays (with tools) that will be needed for an upcoming surgery. Consistent with what was mentioned above, FIG. 47 shows that a combined BT and autoclave tag reader 4710 on the torso of one such robot 4700 may be used to read an autoclave and/or BT tag of a certain tray in the SPD via the tag's antenna(s) 4720 as incorporated into the blue wrapping 4730 of a particular tray 4740. As additional conformation, the robot 4700 may use a camera 4750 in its eye socket to image the tray's barcode 4760 and confirm that the barcode identifier matches the identifier read via the wireless signals from the antenna 4720 for redundancy, helping to ensure the correct tray is being taken off the shelf and loaded onto the case cart. An output 4770 might also be presented internally to the robot's processor system to indicate the match along with the tray's unique identifier.

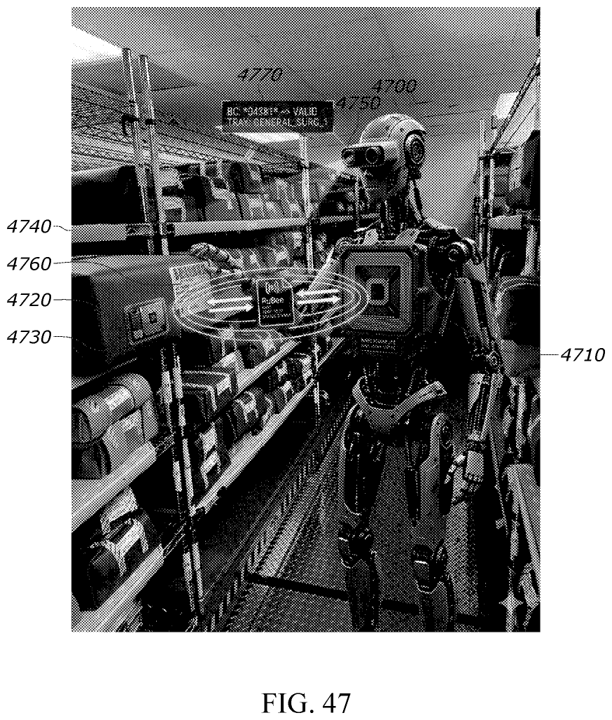

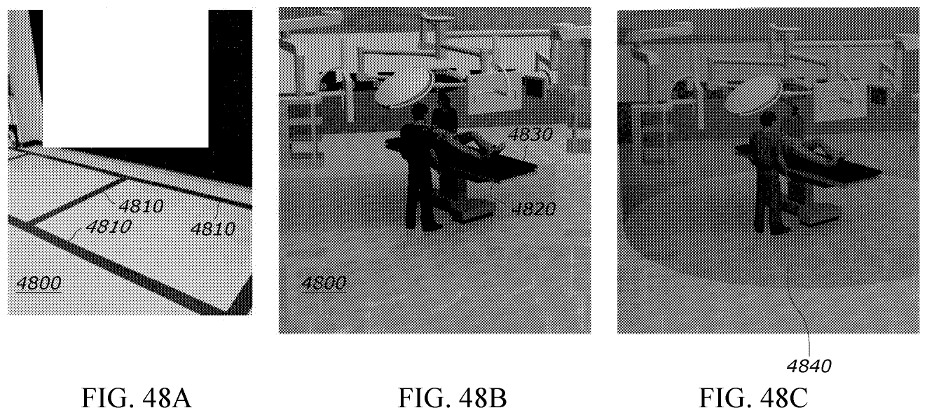

Turning now to another aspect that may be implemented consistent with present principles, it is to be understood that an operating room may also be equipped with readers and antennas for reading IEEE 1902.1 transmissions, BT transmissions, and other types of wireless signals as disclosed herein. Accordingly, as shown in FIG. 48A, a floor 4800 of the OR may be wired with antennas 4810 secured to the floor 4800 via tape or other means. FIG. 48B shows that antennas 4820 may also be attached to the surgical bed 4830 of the OR as well as to the OR's door, ceiling, etc.

FIG. 48C then shows a volumetric representation 4840 of the sensing range of the antennas around the surgery bed 4830. The antennas may thus be used to interrogate autoclave tags/RuBee tags on trays, medical instruments, personnel, and even surgery sponges so that their current location can be identified to make sure the sponges or surgery other items are not retained in the patient at the end of the surgery. For example, a GUI might be presented on a display in the OR to indicate the current locations of all sponges to ensure they are located elsewhere and are not still in the patient. A reader like the reader 4200 or 4710 may also be used to confirm the locations of all items used for the surgery are no longer in the patient.

A system herein may include server and client components which may be connected over a network such that data may be exchanged between the client and server components. The client components may include one or more computing devices including mobile smart phones and other mobile devices, wearable devices such as smart glasses and smart watches, wearable devices in the form of extended reality (XR) headsets beyond smart glasses that can nonetheless present stereoscopic images thereon (such as virtual reality (VR) headsets and augmented reality (AR) headsets), display devices such as televisions (e.g., smart TVs, Internet-enabled TVs), personal computers such as laptops, desktop, and tablet computers, and still other types of client devices including but not limited to Internet of things (IoT) devices. These client devices may operate with a variety of operating environments. For example, client devices consistent with present principles may employ, as examples, Linux operating systems, operating systems from Microsoft, or a Unix operating system, or operating systems produced by Apple, Inc. or Google. These operating environments may be used to execute one or more browsing programs, such as a browser made by Microsoft, Apple, Google, or Mozilla or other browser program that can access websites and servers such as those discussed herein. The operating environments may also be used to execute other (e.g., Internet-networked) dedicated mobile applications (“apps”) that can access websites and servers over a network such as the Internet, a local intranet, or a virtual private network.

For any robotic tasks discussed herein, a respective robot may be used as dedicated to that task. In other embodiments a single robot may perform two or more of the robotic tasks discussed above.

Servers and/or gateways may be used that may include one or more processors executing instructions that configure the servers to receive and transmit data over a network such as the Internet. Or a client and server can be connected over a local intranet or a virtual private network. A server or controller may be instantiated by a personal computer, mobile device, rack or blade server, etc.

Information may be exchanged over a network between client devices and servers. To this end and for security, servers and/or clients can include firewalls, load balancers, temporary storages, and proxies, and other network infrastructure for reliability and security.

As used herein, instructions may refer to computer-implemented steps for processing information in the system. Instructions can be implemented in software, firmware or hardware, or combinations thereof and include any type of programmed steps undertaken by components of the system.

A processor may be a single-or multi-chip processor that can execute logic by means of various lines such as address lines, data lines, control lines, registers, and shift registers. Moreover, any logical blocks, modules, and circuits described below can be implemented or performed with a processor/processor system such as a central processing unit (CPU), a digital signal processor (DSP), a field programmable gate array (FPGA) or other programmable logic device, an application specific integrated circuit (ASIC), discrete gate or transistor logic, discrete hardware components, or any combination thereof designed to perform the functions described herein. A processor can be implemented by a controller or state machine or a combination of computing devices.

Software modules described by way of the flow charts and user interfaces herein can include various sub-routines, procedures, etc. Without limiting the disclosure, logic stated to be executed by a particular module can be redistributed to other software modules and/or combined together in a single module and/or made available in a shareable library.

The functions and methods described below, when implemented in software, can be written in an appropriate language such as but not limited to hypertext markup language (HTML)-5, Java®/Javascript, C # or C++, and can be stored on or transmitted from a computer-readable storage medium such as a hard disk drive (HDD) or solid state drive (SSD), random access memory (RAM), read-only memory (ROM), electrically erasable programmable read-only memory (EEPROM), compact disk read-only memory (CD-ROM) or other optical disk storage such as digital versatile disc (DVD), magnetic disk storage or other magnetic storage devices including removable thumb drives, etc. A connection may establish a computer-readable medium. Such connections can include, as examples, hard-wired cables including fiber optics and coaxial wires and digital subscriber line (DSL) and twisted pair wires.

In an example, a processor system can access information over its input lines from data storage, such as a computer readable storage medium as referenced above, and/or the processor system can access information wirelessly from an Internet server by activating a wireless transceiver to send and receive data. Data typically is converted from analog signals to digital by circuitry between the antenna and the registers of the processor system when being received and from digital to analog when being transmitted. The processor system then processes the data through its shift registers to output calculated data on output lines, for presentation of the calculated data on the device, etc.

Components included in one embodiment can be used in other embodiments in any appropriate combination. For example, any of the various components described herein and/or depicted in the Figures may be combined, interchanged, or excluded from other embodiments. “A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together.

The term “a” or “an” in reference to an entity refers to one or more of that entity. As such, the terms “a” or “an”, “one or more”, and “at least one” can be used interchangeably herein.

The term “circuit” or “circuitry” may be used in the summary, description, and/or claims. The term “circuitry” includes all levels of available integration, e.g., from discrete logic circuits to the highest level of circuit integration such as VLSI, and includes programmable logic components programmed to perform the functions of an embodiment as well as processors (e.g., special-purpose processors) programmed with instructions to perform those functions.

Network or communication interfaces can be provided on any component described herein for communication over at least one network such as the Internet, a WAN, a LAN, etc. under control of one or more processors of a processor system. These interfaces may be, without limitation, a Wi-Fi transceiver and/or wireless telephony transceiver for communicating over a wireless cellular network (e.g., operated by Verizon, T-Mobile, or AT&T), both of which are examples of a wireless computer network interface. These interfaces may also include, as additional examples, a mesh network transceiver, a wired or wireless modem or router, or other appropriate interface.

A processor system may include one or more processors acting independently or in concert with each other to execute an algorithm (e.g., the algorithms referenced herein), whether those processors are in one device or more than one device. Thus, in some specific examples, a processor system may include a single processor, while in other examples a processor system may include more than one processor. In either case, the processor system may control the devices disclosed herein to undertake present principles.

Computer memories/computer-readable storage media include disk-based or solid-state storage that are not transitory signals. Cameras may include thermal imaging camera, digital cameras such as red-green-blue cameras like webcams, infrared (IR) cameras/sensors, and/or other types of cameras or other optical sensors in communication with a processor system. Cameras may be used to gather still images (e.g., single image frames) and/or video with plural image frames consistent with present principles. Any component herein may include a Bluetooth® transceiver and other Near Field Communication (NFC) element for communication with other devices using respective Bluetooth and/or NFC wireless technologies/communication standards. An example NFC element can be a radio frequency identification (RFID) element.

Touch-enabled display(s) consistent with present principles may include, for example, a capacitive or resistive touch sensing layer with a grid of electrodes for touch sensing consistent with present principles (e.g., to provide input to the graphical user interfaces (GUIs) discussed herein).

A battery may be provided for powering any devices disclosed here, as may alternating current (AC) power sources such as wall outlets. A kinetic energy harvester may also be embodied on certain devices to turn kinetic energy into power to charge the battery and/or otherwise power the device.

One or more graphics processing unit (GPUs) may also be included on a devices disclosed herein, including for AI-related processing.

A server may include at least one server processor system, at least one tangible computer readable storage medium such as disk-based or solid-state storage, and at least one network interface that, under control of the server processor system, allows for communication with the other illustrated and/or disclosed devices over the network, and indeed may facilitate communication between servers and client devices in accordance with present principles. Note that the network interface may be, e.g., a wired or wireless modem or router, Wi-Fi or Ethernet transceiver, or other appropriate interface such as, e.g., a wireless telephony transceiver.

Accordingly, in some embodiments a server may be an Internet server or an entire server “farm” of multiple servers. If desired, the server may include and perform “cloud” functions such that the devices of the system may access a “cloud” environment via the server in example embodiments.

The components shown in the figures may include some or all components described above. Any user interfaces (UI) described herein may be consolidated and/or expanded, and UI elements may be mixed and matched between UIs. UIs may be presented at a client device under control of the client device itself and/or under control of a server as remotely controlling the client device to present the UIs thereon. Also note that selectors and options on any UIs discussed herein may be selected via cursor input, touch input to a touch-enabled display on which the UI is presented, using voice input, and/or using other input methods. A graphical user interface (GUI) is one example of a UI, with it being understood that UIs may also include audible UIs and gesture-based UIs.

Present principles may employ various machine learning models, including deep learning models. Machine learning models consistent with present principles may use various algorithms trained in ways that include supervised learning, unsupervised learning, semi-supervised learning, reinforcement learning, feature learning, self-learning, and other forms of learning. Examples of such algorithms, which can be implemented by computer circuitry, include one or more neural networks, such as a convolutional neural network (CNN), a linear classifier, a recurrent neural network (RNN), and a type of RNN known as a long short-term memory (LSTM) network. Generative pre-trained transformers (GPTT) and other large language models (LLM) and more generally generative models (GM) also may be used. Support vector machines (SVM) and Bayesian networks also may be considered to be examples of machine learning models. In addition to the types of networks set forth above, models herein may be implemented by classifiers.

As understood herein, performing machine learning may therefore involve accessing and then training a model on training data to enable the model to process further data to make inferences. The training data may include one or more training sets, one or more validation sets, and one or more test sets.

An artificial neural network/artificial intelligence model trained through machine learning may thus include an input layer, an output layer, and multiple hidden layers in between that are configured and weighted to make inferences about an appropriate output.

In one particular aspect, devices and methods consistent with present principles may operate substantially as shown and described herein but may be claimed as including some but not all aspects in any intermediate generalization.

Before concluding, it is to be understood that although a software application for undertaking present principles may be vended with a device, present principles apply in instances where such an application is downloaded from a server to a device over a network such as the Internet. Furthermore, present principles apply in instances where such an application is included on a computer readable storage medium that is vended and/or provided by itself, where the computer readable storage medium is not a transitory signal and/or a signal per se.

The disclosed concepts are rooted in computer technology for computers to carry out their functions.

While particular example embodiments are herein shown and described in detail, these are not intended to be limiting and it is to be understood that the subject matter which is encompassed by the present invention is limited only by the claims.

What is claimed is:

1. A system, comprising:
a first Bluetooth (BT) tag storing a first identifier associated with a surgical case cart, the first BT tag configured to communicate with a plurality of BT readers spaced apart from each other in a healthcare facility;
a second BT tag storing a second identifier associated with a surgical bed, the second BT tag configured to communicate with the plurality of BT readers; and
a third BT tag storing a third identifier associated with a medical implant, the third BT tag configured to communicate with the plurality of BT readers.

2. The system of claim 1, wherein each of the first, second, and third BT tags comprises a respective motion sensor, and wherein each of the first, second, and third BT tags is configured to broadcast the respective first, second, or third identifier responsive to one or more signals from the respective motion sensor indicating motion of the respective BT tag.

3. The system of claim 2, wherein each respective motion sensor comprises a respective accelerometer.

4. The system of claim 1, wherein each of the first, second, and third BT tags is configured to broadcast the respective first, second, or third identifier at respective predefined intervals.

5. The system of claim 1, comprising:
a processor system; and
storage accessible to the processor system and comprising instructions executable by the processor system to:
identify a respective location of each of the first, second, and third BT tags based on receipt of one or more signals from each respective BT tag; and
present a graphical user interface (GUI) on a display, the GUI indicating the respective locations.

6. The system of claim 5, wherein the instructions are executable to identify the respective locations using one or more of:
one or more channel sounding algorithms;
one or more phased-based ranging algorithms;
one or more round trip timing algorithms; and/or
one or more received signal strength indicator (RSSI) algorithms.

7. The system of claim 5, comprising the display.

8. The system of claim 5, wherein the processor system and storage are embodied on a server associated with the healthcare facility.

9. The system of claim 1, comprising:
a processor system; and
storage accessible to the processor system and comprising instructions executable by the processor system to:
identify a respective location of one or more of the first, second, and third BT tags based on receipt of one or more signals from each respective BT tag;
based on a respective location of one or more of the first, second, and third BT tags, determine a time at which an operation on a patient is to start; and
based on the determination of the time at which the operation is to start, transmit a respective notification to one or more client devices, each respective notification indicating the time at which the operation on the patient is to start.

10. The system of claim 9, wherein the instructions are executable to:
based on respective locations of the first BT tag and the third BT tag, determine the time at which the operation on the patient is to start.

11. The system of claim 10, wherein the time is a first time, and wherein the instructions are executable to:
based on the respective locations of the first BT tag and the third BT tag, determine a second time at which the surgical case cart and the medical implant will be located at an operating room; and
based on the determination of the second time at which the surgical case cart and the medical implant will be located at the operating room, determine the first time at which the operation on the patient is to start.

12. The system of claim 11, wherein the first time is the same as the second time.

13. The system of claim 11, wherein the first time is no more than a threshold amount after the second time.

14. The system of claim 11, wherein the second time is an earliest time at which the surgical case cart and the medical implant will be located at the operating room as determined based on current movement of both the first BT tag and the third BT tag.

15. The system of claim 11, wherein each respective notification indicates an identifier for the operating room.

16. The system of claim 1, wherein the third BT tag is a sticker tag.

17. The system of claim 1, comprising the plurality of BT readers, and further comprising an additional BT reader and cellular communication transceiver disposed on a vehicle that transports the medical implant to the healthcare facility.

18. The system of claim 1, wherein the first BT tag is embodied on a case cart tag that includes both the first BT tag and another tag different from a BT tag, the other tag comprising a transmitter configured to transmit wireless signals in a frequency range of 30-900 kHz.

19. A system, comprising:
a first wireless communication tag storing a first identifier associated with a surgical case cart, the first wireless communication tag configured to communicate with a plurality of readers spaced apart from each other in a healthcare facility;
a second wireless communication tag storing a second identifier associated with a medical implant, the second wireless communication tag configured to communicate with the plurality of readers. a processor system; and
storage accessible to the processor system and comprising instructions executable by the processor system to:
receive one or more first signals from the first wireless communication tag;
receive one or more second signals from the second wireless communication tag;
based on the one or more first signals and the one or more second signals, update an inventory database for the healthcare facility;
receive one or more third signals from the first wireless communication tag;
receive one or more fourth signals from the second wireless communication tag; and
based on the one or more third signals and the one or more fourth signals, manage the scheduling of one or more operations in the healthcare facility.

20. A method, comprising:
receiving one or more first signals from a first wireless communication tag;
receiving one or more second signals from a second wireless communication tag;
based on the one or more first signals and the one or more second signals, updating an inventory database for a healthcare facility;
receiving one or more third signals from the first wireless communication tag;
receiving one or more fourth signals from the second wireless communication tag; and
based on the one or more third signals and the one or more fourth signals, managing the scheduling of one or more operations in the healthcare facility.
